(19) United States
(12) Patent Application Publication
(10) Pub. No.: US 2019/0136292 A1
(43) Pub. Date: May 9, 2019

(54) Universal Calibration Method for Assaying Enzymatic Inhibitors

(71) Applicant: Diagnostica Stago, Gennevilliers (FR)

(72) Inventor: Sébastien Kerdelo, Paris (FR)

(73) Assignee: Diagnostica Stago, Gennevilliers (FR)

(21) Appl. No.: 16/099,889

(22) PCT Filed: May 10, 2017
(86) PCT No.: PCT/FR2017/051112

§ 371 Date: Nov. 8, 2018

(30) Foreign Application Priority Data
May 10, 2016 (FR) 1654148

(51) Int. Cl. C12Q 1/56; G16B 20/00; C12N 9/90; C12N 9/88; C12N 9/14

(52) U.S. Cl. CPC C12Q 1/56; G16B 20/00; C12N 9/90; G01N 2333/96444; C12N 9/14; G01N 2333/811; C12N 9/88

(57) ABSTRACT

The present invention relates to a universal calibration method of use for assaying inhibitors of the same enzyme, for example for assaying inhibitors of an enzyme of blood coagulation. The invention also relates to the use of this universal calibration in a method for assaying a reversible or irreversible inhibitor of the enzyme in a biological sample. The invention also relates to the use of the universal calibration in a method for screening inhibitors of the enzyme.

### PARENT APPLICATION

The present international application claims the priority of the French application number FR 16 54148 filed on May 10, 2016, the content of which is incorporated herein in its entirety by reference.

### FIELD OF THE INVENTION

The present invention relates to a universal calibration method of use for assaying inhibitors of the same enzyme, for example an enzyme of blood coagulation. The invention also relates to the use of this universal calibration in a method for assaying an inhibitor of the enzyme in a biological sample. Since the universal calibration also makes it possible to compare inhibitors with one another, the invention also relates to the use of the universal calibration in a method for screening compounds capable of inhibiting the enzyme.

### CONTEXT OF THE INVENTION

Blood coagulation is a complex physiological phenomenon which involves several factors, especially:
tissue factor, the physiological activator of thrombin generation, and
fibrinogen, converted into fibrin by the activation of thrombin.
   The accumulation of fibrin leads to the formation of a clot which stops the hemorrhage. Clot formation is regulated by a balance between activations and inhibitions, especially involving numerous enzymes and their inhibitors. Upsetting this balance between activations and inhibitions may induce two types of disease: thrombotic diseases and hemorrhagic diseases. With the aim of diagnosing either one of these diseases, or of measuring the activity of therapies established to treat these diseases, it may be beneficial to assay one of the enzymes involved or one of the inhibitors of this enzyme.

An inhibitor of an enzyme of blood coagulation is conventionally assayed by bringing the following into competition for the enzyme: the inhibitor present in the blood sample and a substrate specific to the enzyme. Such assaying is generally achieved using a dedicated kit which is specific to the inhibitor and is based on:
optimized methodology which is generally specific to the inhibitor assay, especially involving dilution of the sample to be tested, specific concentrations of reagents and precise measuring points; and
a mathematical regression model of the calibration curve, which may be linear, polynomial or logarithmic.

This dedicated kit specific to the inhibitor generally comprises:
a calibrator (typically plasmas containing increasing concentration levels of the inhibitor to be assayed); and
quality controls generally containing a low and a high concentration level of inhibitor.

In addition, in cases where it is necessary to be able to carry out assays in parallel, in different samples, of several inhibitors of the same enzyme, the user must have one assay kit per inhibitor. Moreover, for each inhibitor, it is necessary to calibrate the measurement system.

There is therefore a requirement for a method which overcomes all of these restrictions. There is especially a requirement for a method for assaying an inhibitor of an enzyme of blood coagulation, which is easy to carry out and which is not specific to the inhibitor in question, that is to say, in other words, an assaying method which is universal.

### SUMMARY OF THE INVENTION

The invention provides a calibration method that is simple to carry out and relatively inexpensive and that enables parallel assaying of multiple inhibitors of the same enzyme, whether the enzymatic inhibitors are reversible or irreversible. Whereas, in a conventional calibration method, the calibration curve is created from increasing concentrations of the inhibitor that it is desired to assay, in a calibration method according to the invention, the calibration curve is created from decreasing concentrations of the target enzyme. The calibration method according to the invention makes it possible to obtain results expressed as a percentage of anti-enzyme activity, a universal unit common to all the inhibitors of the enzyme, which makes it possible to compare the inhibitory efficiency of these inhibitors on the enzyme. Since the calibration therefore makes it possible to compare inhibitors with one another, the invention also relates to the use of the universal calibration in a screening method for identifying inhibitors of the enzyme. The calibration method according to the invention also makes it possible to measure the amount or the concentration of inhibitor present in a tested sample by producing a conversion chart according to specific mathematical reasoning. A calibration method according to the invention can therefore be applied to the assaying of an inhibitor of the enzyme present in a biological sample, for example to the assaying of a direct oral anticoagulant present in a biological sample originating from a patient treated by the direct oral anticoagulant.

More specifically, the present invention relates to a method for obtaining a universal calibration for assaying an inhibitor of an enzyme, the method comprising the following steps:
a) determining the residual enzymatic activity in the stationary state for each of a plurality of mixtures containing the enzyme E and a substrate S that is labeled and specific to the enzyme, wherein:
   the substrate specific to the enzyme is labeled with a label having a detectable physical property,
   in each of the mixtures, the substrate is present in excess relative to the enzyme,
   the mixtures contain the same initial substrate concentration, [S]₀,
   the mixtures have the same total volume V and the same reaction medium M_{R},
   the mixtures have known and decreasing initial enzyme concentrations, the highest initial enzyme concentration being [E]₀, or have known and decreasing initial enzyme activities, the highest initial enzyme activity being A₀, and
   the residual enzymatic activity in the stationary state of a mixture is determined by the following steps:
      a1) mixing a solution of the enzyme E and a solution of the substrate S to obtain a mixture of known initial enzyme concentration, or of known initial enzyme activity, and of initial substrate concentration [S]₀,
      a2) measuring the value of the detectable physical property of the label and plotting, on a graph, the value of this physical property as a function of time in order to obtain a curve, the curve having a rectilinear portion corresponding to the stationary state, and
      a3) calculating the gradient of the rectilinear portion of the curve obtained in a2), wherein the gradient obtained in a3) is the residual enzymatic activity in the stationary state of the mixture;
b) for each of the mixtures of step a), converting the initial enzyme concentration of the mixture into anti-enzyme activity expressed as a percentage by standardizing said initial enzyme concentration of the mixture relative to the highest initial enzyme concentration [E]₀, or converting the initial enzyme activity of the mixture into anti-enzyme activity expressed as a percentage by standardizing said initial enzyme activity of the mixture relative to the highest initial enzyme activity A₀,
c) creating a universal calibration curve by plotting, on a graph, for each mixture, the anti-enzyme activity determined in step b) as a function of the residual enzymatic activity in the stationary state obtained in step a).

In certain embodiments, the enzyme used in the universal calibration method belongs to the class of the hydrolases, to the class of the lyases, or to the class of the isomerases.

In certain embodiments, the inhibitor that it is desired to assay using the calibration method is a reversible direct or indirect inhibitor, or an irreversible direct or indirect inhibitor.

In certain embodiments, in step b) of the universal calibration method, for a mixture with an initial enzyme concentration [E], the anti-enzyme activity expressed as a percentage is calculated by the equation:

AntiEnzyme_{%}=1−([E]/[E]₀),

and, for a mixture with an initial enzyme activity A, the anti-enzyme activity expressed as a percentage is calculated by the equation:

AntiEnzyme_{%}=1−(A/A₀).

In certain embodiments, in step c) of the universal calibration method, the calibration curve is a straight line with the equation:

$${AntiEnzyme}_{( \% )} = 1 - ( \frac{1}{{v}_{0}} \times v )$$

wherein:
AntiEnzyme_{(%)} is the anti-enzyme activity expressed as a percentage,
v is the residual enzymatic activity in the stationary state, and
1/v₀ is the gradient of the calibration curve, and v₀ is the residual enzymatic activity in the stationary state observed in the absence of an inhibitor.

In certain embodiments, the universal calibration method also comprises a step d) consisting in creating a conversion chart specific to an inhibitor of the enzyme E and which makes it possible to convert the anti-enzyme activity, determined for a sample to be tested, into the amount or concentration of inhibitor. Preferably, step d) comprises the following sub-steps:

d1) determining the residual enzymatic activity in the stationary state for at least two standardization mixtures each containing the inhibitor at a known initial concentration, the enzyme E at the initial concentration [E]₀ or at the initial activity A₀, and the labeled substrate S specific to the enzyme at the concentration [S]₀, wherein:
in each of the standardization mixtures, the enzyme is present in excess relative to the inhibitor,
the standardization mixtures have the same volume V′ and the same reaction medium M_{R}, and
the residual enzymatic activity in the stationary state of a mixture is determined by the following steps:
   d1′) mixing the inhibitor with a solution of the enzyme E and a solution of the substrate S in order to obtain a standardization mixture with a known initial concentration of inhibitor,
   d1″) measuring the value of the detectable physical property of the label and plotting, on a graph, the value of this physical property as a function of time in order to obtain a curve, the curve having a rectilinear portion corresponding to the stationary state, and
   d1′″) calculating the gradient of the rectilinear portion of the curve obtained in d1″), wherein the gradient obtained in step d1′″) is the residual enzymatic activity in the stationary state of the standardization mixture;

d2) for each of the standardization mixtures, using the universal calibration curve obtained in step c) of the universal calibration method in order to determine the anti-enzyme activity of the mixture from the residual enzymatic activity in the stationary state measured in step d1′″) for the standardization mixture; and

d3) creating a standard curve or chart, by plotting on a graph, for each standardization mixture, the initial concentration of inhibitor of the standardization mixture as a function of the anti-enzyme activity determined in step d2).

In certain embodiments, the volume V′ is identical to the volume V.

In certain embodiments, step d) also comprises the sub-step d4) consisting in determining the equation of the standard curve by a regression of the pairs (anti-enzyme activity, concentration of inhibitor) plotted on the graph in step d3).

In certain embodiments,

if the inhibitor is a reversible direct inhibitor, the equation of the standard curve is the following equation:

$${[ I ]}_{0} = i \times \frac{{AntiEnzyme}_{( \% )}}{1 - {AntiEnzyme}_{( \% )}} + e \times {AntiEnzyme}_{( \% )}$$

wherein:

AntiEnzyme_{(%)} is the anti-enzyme activity,

[I]₀ is the concentration of inhibitor present in the sample to be tested, and

i and e are two fixed constants specific to the inhibitor; and

if the inhibitor is an irreversible indirect inhibitor, the equation of the standard curve is the following equation:

[I]₀=e×AntiEnzyme_{(%)}

wherein:

AntiEnzyme_{(%)} is the anti-enzyme activity in the sample to be tested,

[I]₀ is the concentration of inhibitor present in the sample, and

e is a fixed constant specific to the inhibitor.

The invention also relates to a method for assaying an inhibitor of an enzyme in a biological sample, the method comprising the following steps:

1) determining the residual enzymatic activity in the stationary state for a mixture to be tested of reaction medium M_{R}, of volume V″, and containing an aliquot of the biological sample containing the inhibitor, the enzyme E at an initial concentration [E]₀ or at an initial activity A₀, and a labeled substrate S specific to the enzyme at an initial concentration [S]₀, wherein:
the substrate specific to the enzyme is labeled with a label having a detectable physical property, and
the residual enzymatic activity in the stationary state of the mixture to be tested is determined by the following steps:
i) mixing the aliquot of the biological sample with a solution of the enzyme E and a solution of the substrate S to obtain a mixture of initial enzyme concentration [E]₀, or of initial enzyme activity A₀, and an initial substrate concentration [S]₀,
ii) measuring the value of the detectable physical property of the label and plotting, on a graph, the value of this physical property as a function of time in order to obtain a curve, the curve having a rectilinear portion corresponding to the stationary state, and
iii) calculating the gradient of the rectilinear portion of the curve obtained in step ii), wherein the gradient obtained in step iii) is the residual enzymatic activity in the stationary state of the mixture to be tested; and

2) using the universal calibration curve obtained in step c) of the calibration method as claimed in any one of claims 1 to 5 in order to determine the anti-enzyme activity of the biological sample from the residual enzymatic activity in the stationary state measured for the mixture to be tested.

In certain embodiments, the biological sample used in an assaying method according to the invention is a sample of blood, of plasma, of platelet-rich plasma, of platelet-poor plasma, or of plasma containing platelet or erythrocyte microparticles or any other cell. In certain preferred embodiments, the biological sample is a platelet-poor plasma sample.

In certain embodiments, the enzyme used in an assaying method according to the invention is an enzyme of blood coagulation selected from coagulation factors, kallikrein and plasmin, preferably selected from factor IIa, factor Xa and plasmin.

In the embodiments wherein the enzyme is an enzyme of blood coagulation, the inhibitor to be assayed by an assaying method according to the invention may be selected from antithrombin, heparin cofactor II, alpha-2-macroglobulin, hirudin, lepirudin, desirudin, rivaroxaban, apixaban, edoxaban, betrixaban, dabigatran, bivalirudin, argatroban, unfractionated heparins, low-molecular-weight heparins, pentasaccharides and danaparoid sodium.

In certain embodiments, the volume V″ is identical to the volume V.

In certain embodiments, the assaying method according to the invention also comprises the following step:

3) converting the anti-enzyme activity expressed as a percentage and obtained in step 2) into a concentration of inhibitor using the chart specific to the inhibitor which was obtained in step d) of the calibration method as claimed in any one of claims 6 to 10.

The present invention also relates to the use of an assaying method according to the invention for estimating the hemorrhagic risk in a patient treated with a direct oral anticoagulant.

The present invention also relates to a method for estimating the hemorrhagic risk in a patient treated with a direct oral anticoagulant, the method comprising the following steps:
determining the amount or the concentration of direct oral anticoagulant in a biological sample from the patient, using an assaying method of the invention;
comparing this amount or concentration with a predetermined threshold; and
deeming there to be a hemorrhagic risk if the amount or concentration of direct oral anticoagulant is greater than the predetermined threshold.

In certain embodiments, the method for estimating the hemorrhagic risk in a patient treated with a direct oral anticoagulant, also comprising the following step:
determining the amount of anti-inhibitor compound to administer to the patient as a function of the amount or concentration of direct oral anticoagulant measured in the biological sample from the patient.

In certain embodiments, the patient treated with the direct oral anticoagulant is a patient suspected of having received an overdose of direct oral anticoagulant or is a patient recently having undergone a change in treatment from an antivitamin K medicament to the direct oral anticoagulant, or is a patient about to undergo a surgical intervention.

The present invention also relates to a screening method for identifying an inhibitor of an enzyme, comprising the following steps:

1) determining the residual enzymatic activity in the stationary state for a mixture of reaction medium M_{R}, of volume V′″, and containing a test compound at an initial concentration [C], the enzyme E at an initial concentration [E]₀ or at an initial activity A₀, and a labeled substrate S specific to the enzyme at an initial concentration [S]₀, wherein:
the substrate specific to the enzyme is labeled with a label having a detectable physical property,
the enzyme is present in the mixture in excess relative to the test compound, and
the residual enzymatic activity in the stationary state of the mixture is determined by the following steps:
i) mixing the test compound with a solution of the enzyme E and a solution of the substrate S to obtain a mixture of initial enzyme concentration [E]₀, or of enzyme activity A₀, an initial substrate concentration [S]₀ and a concentration [C] of test compound,
ii) measuring the value of the detectable physical property of the label and plotting, on a graph, the value of this physical property as a function of time in order to obtain a curve, the curve having a rectilinear portion corresponding to the stationary state, and
iii) calculating the gradient of the rectilinear portion of the curve obtained in step ii), wherein the gradient obtained in step iii) is the residual enzymatic activity in the stationary state of the compound to be tested;

2) using the universal calibration curve obtained in step c) of the universal calibration method in order to determine the anti-enzyme activity of the test compound from the residual enzymatic activity in the stationary state measured for the mixture; and

3) comparing the anti-enzyme activity of the test compound determined in step 2) with the anti-enzyme activity determined under the same conditions for a standard inhibitor of the enzyme at a concentration [C], or comparing the anti-enzyme activity of the test compound determined in step 2) with a predetermined threshold, wherein the test compound is identified as an inhibitor of the enzyme if the anti-enzyme activity of the test compound is greater than the anti-enzyme activity of the standard inhibitor of the enzyme or if the anti-enzyme activity of the test compound is greater than the predetermined threshold.

In certain embodiments, the volume V′″ is identical to the volume V.

In certain embodiments, the enzyme used in a screening method according to the invention belongs to the class of the hydrolases or to the class of the lyases.

Finally, the present invention relates to a first kit for identifying an inhibitor of an enzyme E comprising
the enzyme E,
a labeled substrate S specific to the enzyme, and
instructions for carrying out a screening method according to the invention; and a second kit for assaying at least one of the inhibitors of an enzyme E in a biological sample, the kit comprising
the enzyme E,
a labeled substrate S specific to the enzyme,
at least one inhibitor of the enzyme, and
instructions for carrying out an assaying method according to the invention.

In certain embodiments, the second kit also comprises at least one other inhibitor.

A more detailed description of certain preferred embodiments of the invention is given below.

### DETAILED DESCRIPTION OF THE INVENTION

As mentioned above, the present invention relates to a calibration method for inhibitors of an enzyme and to the use of this universal calibration in methods for assaying inhibitors of the enzyme and in methods for screening for inhibitors of the enzyme.

### I—Universal Calibration Method

### A. Underlying Principle of a Conventional Method for Assaying Enzymatic Inhibitor

A conventional method for assaying an inhibitor of an enzyme involves at least three elements and brings two biochemical reactions into competition with one another. The three elements are the enzyme (E), the inhibitor (I) for which it is desired to measure the amount or the concentration in the sample tested, and a substrate (S), specific to the enzyme, this substrate being labeled (generally, the labeling is chosen such that the enzymatic reaction results in a release of the label, enabling the detection thereof). The two competing biochemical reactions are: the reaction of inhibition of the enzyme by the inhibitor, and the enzymatic reaction between the enzyme and its substrate.

When the inhibitor is a reversible inhibitor, the reaction of inhibition of the enzyme by the reversible inhibitor is as follows:

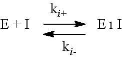

wherein:
E and I denote the enzyme and the inhibitor, as defined above,
E [symbol] I denotes the inactive complex formed between the enzyme and the inhibitor,
kᵢ₊ is the association constant between the enzyme and the reversible inhibitor, and
kᵢ₋ is the dissociation constant between the enzyme and the reversible inhibitor.

When the inhibitor is an irreversible inhibitor, the reaction of inhibition of the enzyme by the irreversible inhibitor is as follows:

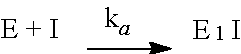

wherein:
E, I and E [symbol] I are as defined above, and
kₐ is the association constant between the enzyme and the irreversible inhibitor.
The enzymatic reaction between the enzyme and its substrate is as follows:

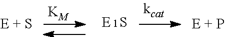

wherein:
E and S denote the enzyme and its labeled substrate as defined above,
E [symbol] S denotes the unstable complex formed by the enzyme and the substrate,
P denotes the product resulting from the catalysis of the substrate by the enzyme,
K_{M} is the Henri-Michaelis-Menten constant, and
k_{cat} is the catalytic constant of the enzyme.

In the cases wherein the inhibitor is indirect, the assay involves a fourth element: a compound A which forms a complex (A [symbol] I) with the inhibitor, which complex is an irreversible inhibitor of the enzyme. Thus, for example, in the case of an irreversible indirect inhibitor, the reactions competing are thus represented in the following scheme:

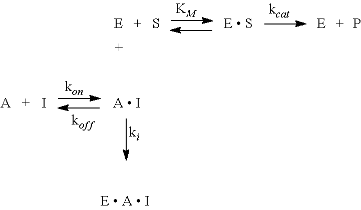

wherein:
E, I, S, K_{M}, and k_{cat} are as defined above;
A is the compound which forms the complex A [symbol] I with the inhibitor;
kₒₙ is the association constant of the complex A [symbol] I;
k_{off} is the dissociation constant of the complex A [symbol] I; and
kᵢ is the association constant of the enzyme and the inhibitor.

In an assaying method, the substrate is initially present in excess relative to the enzyme, which is itself present in excess relative to the inhibitor. During the measurement, the enzyme, inhibited to a greater or lesser extent by the inhibitor, cleaves the substrate to give a product. This results in the release of the label, the appearance and accumulation of which induce observable changes in at least one physical property of the sample tested. Following the variations in this physical property as a function of time by constructing a curve makes it possible to follow the establishment of an equilibrium between the two competing biochemical reactions.

Indeed, after a certain amount of time, the two competing biochemical reactions will reach a local equilibrium referred to as stationary state: that is to say a state wherein the enzyme and the inhibitor (or the enzyme and the complex A [symbol] I for an indirect inhibitor) are in biochemical equilibrium, and wherein the concentrations of enzyme, of inhibitor and of complex E [symbol] I (or of enzyme, of complex A [symbol] I and of complex E [symbol] A [symbol] I, for an indirect inhibitor) are constant. Since the enzyme was initially in excess, at this point in time there remains residual enzymatic activity which may be quantified in order to deduce therefrom the concentration of inhibitor present in the sample tested. Indeed, the residual enzyme activity and the initial concentration of inhibitor are inversely proportional: the higher the concentration of inhibitor present in the sample tested, the more the enzyme is inhibited during the measurement and the less it is active with regard to the substrate during the stationary state; conversely, the lower the concentration of inhibitor present in the sample, the less the enzyme is inhibited during the measurement and the more it is active with regard to the substrate during the stationary state. The relationship between these two values, residual activity of the enzyme in the stationary state and concentration of inhibitor, is thus bijective and there is therefore a unique equivalent residual enzymatic activity for each concentration of inhibitor present in the sample. The stationary state corresponds to the rectilinear portion of the curve presenting variations in the physical property (associated with the release of the label) as a function of time. The gradient of this rectilinear portion is the residual activity of the enzyme which is inversely proportional to the initial concentration of inhibitor.

In a conventional method for assaying an enzymatic inhibitor, a calibration is carried out by determining the residual enzymatic activity in the stationary state for a number n of calibration samples having known concentrations of inhibitor. The calibration curve of a conventional assaying method is created by plotting, on a graph, the n pairs (residual enzymatic activity, concentration of inhibitor) obtained. The mathematical equation for the regression of these n pairs makes it possible to determine, from the residual enzymatic activity measured in the stationary state for the sample tested, the concentration of inhibitor present in the sample tested.

### B. Universal Calibration

The present invention provides a calibration method which does not involve the enzymatic inhibitor but rather a single calibrator which is common to all the inhibitors of the enzyme: the enzyme itself. The principle of the universal calibration according to the invention consists in measuring, for calibration samples containing decreasing initial concentrations of enzyme, the residual enzymatic activity in the stationary state, then in creating a calibration curve by plotting the different pairs obtained (residual enzymatic activity, initial enzyme concentration) on a graph. The equation for the regression of these different pairs makes it possible to convert the residual enzymatic activity measured in the stationary state for the sample tested into enzyme concentration equivalent. Rather than providing the result as enzyme concentration equivalent, the result is provided here as anti-enzyme activity equivalent, expressed as a percentage.

More specifically, the present invention relates to a method for obtaining a universal calibration for assaying an inhibitor of an enzyme, the method comprising the following steps:
a) determining the residual enzymatic activity in the stationary state for each of a plurality of mixtures containing the enzyme E and a substrate S that is labeled and specific to the enzyme, wherein:
   the substrate specific to the enzyme is labeled with a label having a detectable physical property,
   in each of the mixtures, the substrate is present in excess relative to the enzyme,
   the mixtures contain the same initial substrate concentration, [S]₀,
   the mixtures have the same total volume V and the same reaction medium M_{R},
   the mixtures have known and decreasing initial enzyme concentrations, the highest initial enzyme concentration being [E]₀, or have known and decreasing initial enzyme activities, the highest initial enzyme activity being A₀, and
   the residual enzymatic activity in the stationary state of a mixture is determined by the following steps:
      a1) mixing a solution of the enzyme E and a solution of the substrate S to obtain a mixture of known initial enzyme concentration, or of known initial enzyme activity, and of initial substrate concentration [S]₀,
      a2) measuring the value of the detectable physical property of the label and plotting, on a graph, the value of this physical property as a function of time in order to obtain a curve, the curve having a rectilinear portion corresponding to the stationary state, and
      a3) calculating the gradient of the rectilinear portion of the curve obtained in a2),
      wherein the gradient obtained in a3) is the residual enzymatic activity in the stationary state of the mixture;
b) for each of the mixtures of step a), converting the initial enzyme concentration of the mixture into anti-enzyme activity expressed as a percentage by standardizing said initial enzyme concentration of the mixture relative to the highest initial enzyme concentration [E]₀, or converting the initial enzyme activity of the mixture into anti-enzyme activity expressed as a percentage by standardizing said initial enzyme activity of the mixture relative to the highest initial enzyme activity A₀, and
c) creating a universal calibration curve by plotting, on a graph, for each mixture, the anti-enzyme activity determined in step b) as a function of the residual enzymatic activity in the stationary state obtained in step a).

### Step a) of the Universal Calibration Method

Step a) of the universal calibration method according to the invention consists in determining the residual enzymatic activity in the stationary state for each of a plurality of calibration mixtures containing the enzyme to be calibrated and a substrate specific to the enzyme in a reaction medium M_{R}. “Plurality of mixtures” is intended to mean at least 2 different mixtures, preferably at least 4 different mixtures, for example 4, 5, 6, 7, 8 or 9 different mixtures, or else at least 10 different mixtures, for example 10, 11, 12, 13, 14 or 15 different mixtures or more than 15 different calibration mixtures.

Enzymes

An enzyme is a protein or a glycoprotein with catalytic properties. A universal calibration method described here may be applied to any enzyme which, with its specific substrate, is involved in an enzymatic reaction of the following type:

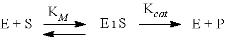

wherein E, S, E S, P, K_{M}, and k_{cat} are as defined above.

More generally speaking, the enzymes to which a calibration method according to the invention may be applied are enzymes for which the enzymatic reaction with the substrate does not involve any coenzyme. Such enzymes are known in the art and belong to the following classes: hydrolases, lyases and isomerases. Thus, the enzyme used in a universal calibration method according to the invention belongs to the class of the hydrolases, to the class of the lyases, or to the class of the isomerases. Preferably, the enzyme used in a calibration method belongs to the class of the hydrolases or to the class of the lyases.

Hydrolases constitute a class of enzymes which catalyze hydrolysis reactions; this class contains esterases which hydrolyze esters, peptidases which hydrolyze oligosaccharides or polysaccharides, and phosphatases which hydrolyze phosphorus-based products. Among the peptidases (or proteases) a distinction is made between aminopeptidases (which sequentially release N-terminal amino acids), carboxypeptidases (which sequentially release C-terminal amino acids), dipeptidases (which hydrolyze dipeptides) and proteinases (which hydrolyze proteins). In the examples presented in the present document, the inventor has used factor Xa as enzyme, which is a protease and therefore belongs to the class of the hydrolases.

Lyases constitute a class of enzymes which catalyze the breakage of different chemical bonds by means other than hydrolysis or oxidation, often forming a new double bond or a new ring. This class of enzymes contains, but is not limited to, lyases which break carbon-carbon bonds such as decarboxylases, aldolases and oxoacid lyases; lyases which break carbon-oxygen bonds such as dehydratases; lyases which break carbon-nitrogen bonds such as phenylalanine ammonia lyase; lyases which break carbon-sulfur bonds, etc.

Isomerases constitute a class of enzymes which catalyze changes within a molecule, often by rearrangement of functional groups and conversion of the molecule into one of the isomers thereof. This class of enzymes contains, but is not limited to, racemases, epimerases, cis-trans isomerases, intramolecular oxidoreductases, intramolecular transferases, intramolecular lyases and topoisomerases.

The enzyme used in a calibration method according to the invention may be an enzyme of bacterial, viral, fungal or plant origin or may be a mammalian enzyme (human or animal). In particular, a universal calibration method according to the present invention may be applied to any enzyme for which it is beneficial to identify inhibitors, for example with the aim of developing human or veterinary therapeutic inhibitors, or else any enzyme for which it is desirable to quantify the presence of an inhibitor in a biological sample, for example the presence of a therapeutic inhibitor in a biological sample originating from a patient treated by the therapeutic inhibitor.

Labeled Substrate Specific to the Enzyme

Before being able to catalyze a chemical reaction, enzymes must firstly bind to their substrates. The terms “substrate” and “enzymatic substrate” are used here interchangeably. They have the meaning known in the art and denote any molecule capable of being subjected to the action of an enzyme. Those skilled in the art will appreciate that the substrate used in a calibration method must be a substrate specific to the enzyme which is the subject of the calibration. Those skilled in the art also know that, when the specificity of the enzyme is broad, that is to say when it tolerates several substrates, the choice of the ideal substrate is actually a compromise between the specificity of this substrate for the enzyme (which is responsible for the exactness of the measurements) and analytical constraints associated with the sensitivity and practicability of the measurements.

The substrate used in a calibration method according to the invention is a labeled substrate having a detectable physical property. The substrate is preferably labeled such that the enzymatic reaction between the enzyme and the substrate results in the release of the label, thereby making it possible to detect this label.

Thus, “labeled substrate” is intended to denote a substrate which contains, or is associated with (for example non-covalently), or is bonded to (for example covalently) a detectable label.

Different types of labeling and labels are well known to those skilled in the art and may be used within the context of the present invention, including chromogenic, fluorescent, chemiluminescent, electrochemical or radioactive labels, etc. In the examples presented in the present document, the inventor has used the enzyme F.Xa (bovine factor Xa) and the chromogenic substrate MAPA-Gly-Arg-pNA, wherein the peptide sequence MAPA-Gly-Arg (4-Methyl-2-Amino-[Methoxy-(Ethoxy)-Carbamate]-Pentanoyl-Glycine-Arginine) is covalently bonded to para-nitroaniline (pNA). The enzymatic reaction releases pNA, the optical density (or absorbance) of which can be monitored in the mixture at a wavelength of between 400 and 500 nm, typically at 405 nm.

Properties of the Calibration Mixtures

The calibration mixtures used in a method according to the invention have known and decreasing initial enzyme concentrations, the highest initial enzyme concentration being [E]₀. Alternatively, the calibration mixtures have known and decreasing initial enzyme activities, the highest initial enzyme activity being A₀.

Those skilled in the art will appreciate that the highest initial enzyme concentration, [E]₀, and the range of initial enzyme concentrations used for the calibration are selected as a function of the intended use of the calibration curve (for example use in a screening method for which the operator fixes the initial concentration of test compound, or use in a method for assaying a therapeutic inhibitor in a biological sample from a patient for which it is known that the concentration of therapeutic inhibitor is very low or very high relative to a predetermined threshold). However, [E]₀ and the range of initial enzyme concentrations must be chosen such that, in a biological sample (or in a screening sample), the enzyme is always present in excess relative to the inhibitor. This excess guarantees a non-zero residual enzymatic activity measurement. “Enzyme present in excess relative to the inhibitor” is intended to mean an initial enzyme concentration at least 1.25 times higher than the concentration of inhibitor that it is desired to assay (or than the concentration of test compound that it is desired to test), preferably at least 1.33 times higher, and more preferably still at least 1.5 times higher. Thus, the initial enzyme concentration in a calibration mixture may be any concentration which meets this condition. Similarly, the highest initial enzymatic activity, A₀, in one of the calibration mixtures is chosen as a function of the intended use of the calibration curve.

With the exception of their initial enzyme concentrations, the calibration mixtures used in a calibration method according to the invention are identical. In particular, they contain the same initial substrate concentration [S]₀. Moreover, in all the calibration mixtures, the substrate is present in excess relative to the enzyme. This excess makes it possible to have a substrate concentration which varies very little during the stationary state. “Substrate present in excess relative to the enzyme” is intended to mean an initial substrate concentration at least 10 times higher than the initial enzyme concentration, preferably at least 100 times higher than the initial enzyme concentration, and more preferably still at least 1000 times higher or at least 10 000 times higher than the initial enzyme concentration. In other words, in a calibration mixture having an initial enzyme concentration [E]ₙ, the enzyme is present in excess relative to the substrate if the [E]ₙ/[S]₀ ratio is greater than or equal to 10, preferably greater than or equal to 100 and more preferably greater than or equal to 1000 or 10 000.

All the calibration mixtures also have the same total volume V. A calibration method may be carried out using calibration mixtures having any total volume V. It is obvious that, for cost reasons, minimizing analytical volumes is always desirable. Thus, for example, the volume V may be between several milliliters (for example 1 ml or 2 ml) and several hundred microliters (for example 500 μl, 400 μl, 300 μl or less than 300 μl).

The calibration mixtures also all have the same reaction medium M_{R}. “Reaction medium” is intended to mean the solvent wherein the inhibition reaction and the enzymatic reaction are brought into competition. The reaction medium may be any reaction medium suitable for such reactions. Given the influence that the reaction medium may have on the enzymatic reactions, those skilled in the art will understand that the calibration and the assay (or the calibration and the screening) must be carried out in the same reaction medium or substantially the same reaction medium. Thus, for example, when the aim is to assay an inhibitor present in a biological sample originating from a patient treated by an inhibitor of the enzyme, the reaction medium of a calibration mixture contains an aliquot of a biological sample from a healthy patient (that is to say a biological sample not containing the inhibitor—see below).

In the examples described at the end of the present document, the calibration was carried out with calibration mixtures containing 50 μl of healthy plasma, 150 μl of substrate solution and 150 μl of enzyme solution for a total volume V of 350 μl. With such a calibration, the assay may be carried out with a mixture to be tested containing 50 μl of plasma obtained from a patient treated by an inhibitor of the enzyme, 150 μl of substrate solution and 150 μl of enzyme solution for a total volume V of 350 μl or more generally with a mixture to be tested containing plasma obtained from a patient treated by an inhibitor of the enzyme, substrate solution, and enzyme solution in the ratio 1:3:3 by volume.

Preparation of the Calibration Mixtures

Each calibration mixture is prepared by mixing a substrate solution and an enzyme solution so as to obtain a mixture with an initial substrate concentration [S]₀ and a known initial enzyme concentration or known initial enzyme activity. It is for example possible to obtain the mixtures by serial dilution of the same stock concentration of enzyme of known concentration. For the serial dilutions, a buffer solution or plasma (see below) may be used.

In the embodiments wherein an aliquot of a healthy biological sample is used, a calibration mixture is prepared by mixing the aliquot (diluted or undiluted) of the healthy biological sample with a solution of the enzyme E and a solution of the substrate S in order to obtain a mixture of known initial enzyme concentration, or known initial activity, and a substrate concentration [S]₀. Those skilled in the art know that the order of addition of the enzyme solution and of the substrate solution to the healthy biological sample is of no importance in the case of direct inhibitors. Indeed, the mixture to be tested may be obtained:
either by firstly mixing the healthy biological sample with the enzyme solution, then by adding the labeled substrate solution (incubation may be carried out before addition of the substrate solution),
or by firstly mixing the healthy biological sample with the labeled substrate solution, then by adding the enzyme solution (incubation may be carried out before addition of the enzyme solution).
   However, in the case of indirect inhibitors, the healthy biological sample is first mixed with the solution of the enzyme in excess, and the mixture is incubated for a sufficient amount of time to enable the phase of reaction equilibrium to be reached (that is to say the phase in which all of the complex A [symbol] I is fixed to the enzyme). The substrate solution is then added at the end of the incubation period (see example 2) and initiates the enzymatic reaction.

Experimental Conditions

The inhibition reaction and the enzymatic reaction may be carried out under any suitable physicochemical experimental conditions (pH, temperature, ionic strength, etc.). However, those skilled in the art will appreciate that the reactions of all the calibration mixtures must be carried out under the same experimental conditions (in addition to being carried out in the same, or substantially the same, reaction medium).

The optimal experimental conditions of an enzymatic reaction are known in the art or may be readily determined by those skilled in the art. In the examples presented here, the inventor used a temperature between 35° C. and 40° C., and a reaction medium containing Owren Koller buffer (pH 7.35) and/or plasma.

Determining the Residual Enzymatic Activity in the Stationary State

As indicated above, monitoring the changes in the detectable physical property of the label as a function of time makes it possible to monitor the establishment of stationary equilibrium. The monitoring is carried out by measuring the value of the detectable physical property of the label as a function of time. The nature of the label determines the technique used to measure the physical property. Thus, if the label is a chromogenic label, the physical property is optical density (or absorbance) which may be measured by spectrophotometry; if the label is a fluorescent label, the physical property is measured by spectrofluorimetry, etc.

Changes in the detectable physical property of the label are monitored either for a sufficiently long period of time to reach and observe stationary equilibrium, or merely for the period of stationary equilibrium (for example when the time to reach such an equilibrium is known or has been determined beforehand). More specifically, by plotting the value of the physical property as a function of time on a graph, the curve obtained has a rectilinear portion after a certain amount of time, which portion corresponds to the stationary state. Those skilled in the art therefore know how to determine the optimal duration for monitoring changes in the physical property of the label. In the example presented below which concerns direct oral anticoagulants (DOAs), the inventor mixed the sample containing the DOA with the substrate solution and incubated the mixture obtained for 240 seconds, then added the enzyme solution, which initiates the enzymatic reaction. The reaction was monitored by measuring the optical density of the label pNA at 405 nm over time for a duration of 30 seconds starting 50 seconds after the initiation of the enzymatic reaction, i.e., between 50 and 80 seconds. In the example presented below which concerns heparins, the inventor mixed the sample containing heparin with the enzyme solution and incubated the mixture obtained for 11.5 minutes, then added the substrate solution, which initiates the enzymatic reaction. The reaction was monitored by measuring the optical density of the label pNA at 405 nm for 30 seconds starting 20 seconds after the initiation of the enzymatic reaction.

A curve for monitoring the labeling as a function of time is obtained for each calibration mixture.

A calibration method according to the invention subsequently comprises a step consisting in calculating, for each mixture, the gradient of the rectilinear portion of the curve of the changes in the detectable physical property of the label as a function of time. For a given calibration mixture, that is to say for a given initial enzyme concentration, the residual enzymatic activity in the stationary state is the gradient of the rectilinear portion of the curve created for the mixture.

### Step b) of the Universal Calibration Method

Step b) of a universal calibration method according to the invention consists in, for each of the calibration mixtures, converting the initial enzyme concentration of the mixture into anti-enzyme activity expressed as a percentage by standardizing said initial enzyme concentration of the mixture relative to the highest initial enzyme concentration [E]₀, or in converting the initial enzyme activity of the mixture into anti-enzyme activity expressed as a percentage by standardizing said initial enzyme activity of the mixture relative to the highest initial enzyme activity A₀.

The standardization may be carried out by any method. However, preferably, for a mixture having an initial enzyme concentration [E] or an initial enzyme activity A, the anti-enzyme activity, expressed as a percentage for this mixture, AntiEnzyme_{(%)}, is calculated by one of the following equations:

AntiEnzyme_{(%)}=1−([E]/[E]₀), or AntiEnzyme_{(%)}=1−(A/A₀).

Thus, for the sample having the highest initial enzyme concentration [E]₀ or the highest initial activity A₀, the anti-enzyme activity, AntiEnzyme_{(%)}, is zero.

For example, if the highest enzyme concentration [E]₀ is 10 nM, then the associated anti-enzyme activity is 0%. In the case wherein 4 measurements are carried out, the 3 remaining measurements may be obtained for concentrations of 7.5 nM, 5 nM and 2.5 nM; their associated anti-enzyme activities, calculated as indicated above, are 0.25 (i.e. 25%), 0.5 (i.e. 50%) and 0.75 (i.e. 25%), respectively.

### Step c) of the Universal Calibration Method

Step c) of a universal calibration method according to the invention consists in creating the calibration curve by plotting, on a graph, for each calibration mixture, the anti-enzyme activity calculated in step b) as a function of the residual enzymatic activity in the stationary state obtained in step a).

The equation of the calibration curve may be calculated by regression of the pairs (anti-enzyme activity, residual enzymatic activity in the stationary state) plotted on the graph. The regression may be a linear or polynomial regression. In the case wherein the regression is linear, the equation of the calibration curve is “equation 1” which is of the form:

$${AntiEnzyme}_{( \% )} = 1 - ( \frac{1}{{v}_{0}} \times v )$$

wherein:
AntiEnzyme_{(%)} is the anti-enzyme activity expressed as a percentage,
v is the residual enzymatic activity measured in the stationary state, and
1/v₀ is the gradient of the calibration curve, and v₀ corresponds to the residual enzymatic activity measured in the stationary state, observed when the concentration of inhibitor present in the sample is zero (i.e. the maximum residual enzymatic activity).

The results are expressed as a percentage: the unit is thus common to all the inhibitors of the enzyme and makes it possible to compare their inhibitory action with one another.

### Chart

As indicated above, a calibration method may be intended to be used in the assaying of an inhibitor of the enzyme. “Inhibitor of the enzyme” or “enzymatic inhibitor” denotes any molecule which binds to the enzyme and decreases the activity thereof. The inhibitor may be reversible or irreversible. “Reversible inhibitor” is intended to mean an inhibitor which associates with the enzyme non-covalently (for example via hydrogen bonds, hydrophobic interactions and/or ionic bonds), which inactivates the enzyme in a non-permanent manner. Reference is made here to a “reversible direct inhibitor” if the inhibitor I associates directly with the enzyme, and to a “reversible indirect inhibitor” if A [symbol] I, the complex of the inhibitor with the compound present in the biological sample, associates with the enzyme. “Irreversible inhibitor” of an enzyme is intended to mean an inhibitor which associates with the enzyme via at least one covalent bond and thus forms a stable complex with the enzyme, which inactivates the enzyme permanently. Reference is made to a “reversible direct inhibitor” if the inhibitor I associates directly with the enzyme, and to a “reversible indirect inhibitor” if A [symbol] I, a complex of the inhibitor with a natural inhibitor of the enzyme present in the biological sample, associates with the enzyme.

With the aim of providing the result of an assay not in terms of anti-enzyme activity expressed as a percentage but in the form of an amount or of a concentration of the inhibitor (for example in ng/ml), which corresponds to a strict quantitative unit, a calibration method according to the invention may also comprise an additional step of creating a chart. Thus, in certain embodiments, the calibration method according to the invention also comprises a step d) consisting in creating a conversion chart specific to an inhibitor of the enzyme E, which makes it possible to convert the anti-enzyme activity, determined for a sample to be tested, into the amount or concentration of inhibitor (for example as concentration expressed in ng/ml).

Preferably, the calibration method according to the invention also comprises a step d) consisting in creating at least one chart specific to an inhibitor of the enzyme E, step d) comprising the following steps:
d1) determining the residual enzymatic activity in the stationary state for at least two standardization mixtures each containing the inhibitor at a known initial concentration, the enzyme E at the concentration [E]₀ and the labeled substrate S specific to the enzyme at the concentration [S]₀, wherein:
   in each of the standardization mixtures, the enzyme is present in excess relative to the inhibitor,
   the standardization mixtures have the same volume V′ and the same reaction medium M_{R}, and
   the residual enzymatic activity in the stationary state of a mixture is determined by the following steps:
      d1′) mixing the inhibitor with a solution of the enzyme E and a solution of the substrate S in order to obtain a standardization mixture with a known initial concentration of inhibitor,
      d1″) measuring the value of the detectable physical property of the label and plotting, on a graph, the value of this physical property as a function of time in order to obtain a curve, the curve having a rectilinear portion corresponding to the stationary state, and
      d1′″) calculating the gradient of the rectilinear portion of the curve obtained in d1″),
      wherein the gradient obtained in step d1′″) is the residual enzymatic activity in the stationary state of the standardization mixture;
d2) for each of the standardization mixtures, using the universal calibration curve obtained in step c) of the universal calibration method in order to determine the anti-enzyme activity of the mixture from the residual enzymatic activity in the stationary state measured in step d1′″) for the standardization mixture; and
d3) creating a standard curve or chart, by plotting on a graph, for each standardization mixture, the initial concentration of inhibitor of the standardization mixture as a function of the anti-enzyme activity determined in step d2).

In certain embodiments, the method may also comprise an additional step d4) consisting in determining the equation of the standard curve by a regression of the pairs (anti-enzyme activity, concentration of inhibitor) plotted on the graph. The regression may be a linear, polynomial or other regression.

Step d1) of the Calibration Method

Step d1) of the calibration method according to the invention consists in determining the residual enzymatic activity in the stationary state for at least two standardization mixtures each containing a known concentration of inhibitor, the enzyme E at the concentration [E]₀ (that is to say identical to the highest initial enzyme concentration used in steps a)-d) of the calibration method), or at the initial activity A₀ (that is to say identical to the initial enzyme activity used in steps a)-d) of the calibration method) and the substrate S at the concentration [S]₀ (that is to say identical to the initial substrate concentration used in steps a)-d) of the calibration method). In the standardization mixtures, the enzyme is present in excess relative to the inhibitor (see above).

For example, the standardization mixtures used to create the chart may have increasing initial concentrations of inhibitor, typically of between 0 and 600 ng/ml. For example, the standardization mixtures used may have initial concentrations equal to 0, 100, 200, 300, 400 and optionally 500 ng/ml. Alternatively, standardization mixtures having concentrations of between 0 and 200 ng/ml may be used. Typically, said concentrations may be equal to 0, 30, 65 and 100 ng/ml; or else equal to 0, 50, 100 and 150 ng/ml.

The standardization mixtures used in step d) of the calibration method have the same volume V′ which may or may not be identical to the volume V of the mixtures used in steps a)-c) of the calibration method. Preferably, the volume V′ is identical to the volume V. The reaction medium M_{R} of the standardization samples is preferably identical or substantially identical to the reaction medium of the samples used in steps a)-c) of the calibration method.

Generally speaking, for the creation of the chart, the inhibition reaction and the enzymatic reaction are brought into competition under any suitable physicochemical experimental conditions (pH, temperature, ionic strength, etc.), identical to those used in the universal calibration method.

The operating conditions described for step a) of the universal calibration are applicable to step d1), in particular regarding the order of the mixtures (see above). Before mixing, the inhibitor is preferably in the form of a solution of inhibitor of known concentration (for example, the inhibitor is added to a healthy biological sample). Step d1″) (measuring the value of the detectable physical property of the label as a function of time and creating a curve having a rectilinear portion) and step d1′″) (calculating the gradient of the rectilinear portion of this curve in order to obtain the residual enzymatic activity in the stationary state) are carried out like steps a2) and a3) of the calibration method.

Step d2) of the Calibration Method

Step d2) of the calibration method according to the invention consists in using the universal calibration curve obtained in step c) of the universal calibration method in order to determine, for each of the standardization mixtures, the anti-enzyme activity of the mixture from the residual enzymatic activity in the stationary state measured for the standardization mixture. This may be determined manually by plotting the residual enzymatic activity in the stationary state measured for a standardization mixture on the graph and by determining the associated anti-enzyme activity. Alternatively, the anti-enzyme activity of a standardization mixture may be calculated using the equation of the calibration curve, for example equation 1, if the calibration curve is a straight line.

Steps d3) and d4) of the Calibration Method

Step d3) of the calibration method according to the invention consists in creating a standard curve or chart, by plotting on a graph, for each standardization mixture, the initial concentration of inhibitor of the standardization mixture as a function of the anti-enzyme activity determined in step d2).

The calibration method according to the invention may also comprise an additional step d4) consisting in determining the equation of the standard curve by a regression of the pairs (anti-enzyme activity, concentration of inhibitor) plotted on the graph in step d3). The regression may be a linear, polynomial or other regression. Those skilled in the art know how to determine the equation of a standard curve.

In certain embodiments of the invention, if the inhibitor is a reversible direct inhibitor, the chart is obtained by carrying out a non-linear regression of the following equation (“equation 2”) on the standardization samples for which the pairs (anti-enzyme activity, concentration of inhibitor) have been determined:

$${[ I ]}_{0} = i \times \frac{{AntiEnzyme}_{( \% )}}{1 - {AntiEnzyme}_{( \% )}} + e \times {AntiEnzyme}_{( \% )}$$

wherein:
AntiEnzyme_{(%)} is the anti-enzyme activity,
[I]₀ is the concentration of inhibitor present in the sample, and
i and e are two fixed constants specific to each inhibitor.
Alternative modeling, for example a polynomial, may also be used.

In other embodiments of the invention, if the inhibitor is an irreversible indirect inhibitor forming a complex A [symbol] I with a compound A present in the biological sample, the chart is obtained by carrying out:

a linear regression of the following equation (“equation 3”) on the standardization samples for which the pairs (anti-enzyme activity, concentration of inhibitor) have been determined:

[I]₀=e×AntiEnzyme_{(%)}

wherein:
AntiEnzyme_{(%)} is the anti-enzyme activity,
[I]₀ is the concentration of inhibitor present in the sample, and
e is a fixed constant specific to each inhibitor,
equation 3 only being valid if the initial concentration of inhibitor is less than or equal to the initial concentration of compound A in each of the standardization mixtures. Alternative modeling, for example a polynomial, may also be used.

Step d) of the calibration method may be repeated for any inhibitor of the enzyme for which it is desired to have a specific chart.

### C. Automation

In certain embodiments, certain steps of the universal calibration method (and also the assaying or screening method) according to the invention are carried out on an automated diagnostic device. Preferably, all the steps of the universal calibration method (and also the assaying or screening method) according to the invention are carried out on such an automated device. For example, as described in the examples presented here, the universal calibration method and the assaying or screening method according to the invention are carried out on the STA-R® Evolution Expert Series automated device from Stago. Such an automated device makes it possible to simultaneously load samples, to carry out mixtures and incubation, and to measure the optical densities. This results in a rapid, reliable and reproducible calibration or assaying method.

A method according to the invention may also be used on a remote biology device or a portable device at the patient's bed.

### II—Uses of the Universal Calibration Method

As indicated above, a calibration curve obtained by a universal calibration method according to the invention is specific to the enzyme and may be used to assay an inhibitor of the enzyme in a biological sample and to identify a compound capable of inhibiting the enzyme in a screening method.

### A. Method for Assaying Enzymatic Inhibitors in a Biological Sample

A method for assaying an inhibitor of an enzyme in a biological sample comprises the following steps:
1) determining the residual enzymatic activity in the stationary state for a mixture to be tested of reaction medium M_{R}, of volume V″, and containing an aliquot of the biological sample containing the inhibitor, the enzyme E at an initial concentration [E]₀ or at an initial activity A₀, and a labeled substrate S specific to the enzyme at an initial concentration [S]₀, wherein:
   the substrate specific to the enzyme is labeled with a label having a detectable physical property, and
   the residual enzymatic activity in the stationary state of the mixture to be tested is determined by the following steps:
      i) mixing the aliquot of the biological sample with a solution of the enzyme E and a solution of the substrate S to obtain a mixture of initial enzyme concentration [E]₀, or of initial enzyme activity A₀, and an initial substrate concentration [S]₀,
      ii) measuring the value of the detectable physical property of the label and plotting, on a graph, the value of this physical property as a function of time in order to obtain a curve, the curve having a rectilinear portion corresponding to the stationary state, and
      iii) calculating the gradient of the rectilinear portion of the curve obtained in step ii),
      wherein the gradient obtained in step iii) is the residual enzymatic activity in the stationary state of the mixture to be tested; and
2) using the universal calibration curve obtained in step c) of the universal calibration method in order to determine the anti-enzyme activity of the biological sample from the residual enzymatic activity in the stationary state measured for the mixture to be tested.

Preferably, the assaying method according to the invention also comprises a step 3) consisting in converting the anti-enzyme activity expressed as a percentage and obtained in step 2) into a concentration of inhibitor using the chart (or standard curve) specific to the inhibitor, obtained in step d) of the universal calibration method. Indeed, the assaying method according to the invention makes it possible to assay an inhibitor:
either in the form of an activity expressed as a percentage: this corresponds to a universal unit which makes it possible to compare the action of different inhibitors with one another, relative to a given enzyme;
or in the form of an amount or concentration (for example in ng/ml): this corresponds to a strict quantitative unit.

### Step 1) of the Assaying Method

Step 1) of the assaying method according to the invention consists in determining the residual enzymatic activity in the stationary state for a mixture containing an aliquot of the biological sample to be tested, the same enzyme E as that used in the calibration method, and the same labeled specific substrate S as that used in the calibration method. In the mixture to be tested, the initial substrate concentration is [S]₀, that is to say the same concentration as that used in the calibration method, and the initial enzyme concentration is [E]₀, that is to say the highest initial enzyme concentration used in the calibration method, or the initial enzyme activity is A₀, that is to say the highest initial enzyme activity used in the calibration method. In a mixture to be tested, the substrate is present in excess relative to the enzyme, which is itself present in excess relative to the inhibitor to be assayed (see above).

The volume V″ of the sample to be tested may or may not be identical to the volume V of the mixtures used in steps a)-c) of the calibration method, and may or may not be identical to the volume V′ of the standardization mixtures used in step d) of the calibration method. Preferably, the volume V″ is identical to the volume V and to the volume V′. The reaction medium of the mixture to be tested is preferably identical or substantially identical to the reaction medium of the calibration samples and of the standardization samples.

Enzymes and Inhibitors

As indicated above, the present invention may be applied to any enzyme (as defined above) for which it is desirable to quantify the presence of an inhibitor, for example a therapeutic inhibitor, in a biological sample originating from a patient treated by the therapeutic inhibitor.

In certain embodiments of the invention, the enzyme used in an assaying method is an enzyme of blood coagulation. “Enzyme of blood coagulation” is intended to mean any enzyme involved in the coagulation of blood, such as, for example, coagulation factors, kallikrein or plasmin. The enzyme of blood coagulation is preferably a mammalian enzyme, preferably a bovine or human enzyme. The enzyme may be of recombinant or plasma origin; it may be purified or unpurified. In certain embodiments, the enzyme of blood coagulation is selected from factor IIa, factor Xa and plasmin.

An irreversible direct inhibitor of an enzyme of blood coagulation that may be assayed by a method according to the invention may be selected from antithrombin, heparin cofactor II, alpha-2-macroglobulin, hirudin, lepirudin and desirudin. A reversible direct inhibitor of an enzyme of blood coagulation that may be assayed by a method according to the invention may be selected from rivaroxaban, apixaban, edoxaban, betrixaban, dabigatran, bivalirudin and argatroban. An irreversible indirect inhibitor of an enzyme of blood coagulation that may be assayed by a method according to the invention may be selected from unfractionated heparins, low-molecular-weight heparins, pentasaccharides such as fondaparinux and danaparoid sodium.

In other embodiments of the invention, the enzyme is an angiotensin-converting enzyme (ACE). ACE is a metalloenzyme belonging to the family of carboxypeptidases. It catalyzes the cleavage of angiotensin I to give angiotensin II, a potent vasoconstrictor. ACE is also involved in the inactivation of bradykinin, a potent vasodilator. The inhibitors of this enzyme constitute an original therapeutic class. They form a recent therapeutic group that play an important role in the treatment of arterial hypertension, cardiac insufficiency and diabetic nephrology. Examples of ACE inhibitors that are used clinically and that may be assayed by a method according to the invention include, but are not limited to, benazepril, captopril, enalapril, monopril, lisinopril, moexipril, perindopril, quinapril, ramipril and tradolapril. The ACE is calibrated using an artificial substrate such as hippuryl-histidyl-leucine (HHL) or 3-(2-furylacryloyl)-L-phenylalanyl-glycyl-glycine (FAPGG). In the latter case, the reduction in the absorbance at 340 nm is proportional to the amount of substrate hydrolyzed.

Biological Samples

The method for assaying an enzymatic inhibitor is carried out on a biological sample, in particular a biological sample originating from a patient.

The term “patient” as used here denotes a human. The term “patient” does not denote a particular age and therefore encompasses children, adolescents and adults. Generally speaking, in an assaying method according to the invention, the patient is a subject who is receiving a treatment based on the enzymatic inhibitor for which it is desired to determine the amount in the biological sample. In the context of the present invention, reference is made to “normal patient” or “healthy patient” when the patient is not being administered the enzymatic inhibitor or any other medicament that may have an effect (of activation or of inhibition) on the enzyme inhibited by the enzymatic inhibitor. In the case of a method for assaying an inhibitor of an enzyme of blood coagulation, the healthy patient is a patient who is not undergoing procoagulant or anticoagulant treatment.

The term “biological sample” is used here in its broadest sense. A biological sample may be any biological fluid wherein the inhibitor is present and may be assayed. Examples of biological fluids that may be used in an assaying method according to the invention include, but are not limited to, blood, serum, plasma, urine, saliva, gastric fluids, sweat, etc. In certain preferred embodiments, an assaying method according to the invention uses a simple blood biological sample from the patient. The blood biological sample may be a sample of blood, of plasma, of platelet-rich plasma, of platelet-poor plasma, or of plasma containing platelet or erythrocyte microparticles or any other cell.

In certain embodiments, the assaying method according to the invention is carried out on a whole blood sample (that is to say blood with all its constituents). The whole blood may be citrated. In this case, the whole blood taken by blood sampling is collected in a citrated tube.

In other embodiments, the assaying method according to the invention is carried out on a plasma sample obtained from the blood sample. The methods for obtaining plasma from human blood are known in the art. Preferably, the biological sample is a platelet-poor plasma (PPP) sample. In this case, it may especially be obtained by centrifugation of the citrated tube comprising the patient's blood sample, for 15 minutes, at a speed of from 2000 to 2500 g, in a thermostated centrifuge at a temperature of between 18 and 22° C. If the PPP sample must be stored, it is possible to use the following protocol, which consists in:
rapidly decanting the plasma, leaving approximately 0.5 cm of plasma above the cell layer of white blood cells and platelets;
recovering the plasma in a hemolysis tube or a plastic tube;
centrifuging this tube again for 15 minutes, at a speed of from 2000 to 2500 g, in a thermostated centrifuge at a temperature of between 18 and 22° C.;
aliquoting in fractions of from 0.5 to 1 mL without taking the bottom of the tube (which contains the cell debris); then
rapidly freezing at a temperature of between −70° C. and −90° C., preferably at −80° C.

The assaying according to the invention can be carried out on any appropriate volume of biological sample. Generally, the aliquot of the biological sample is used in the present invention in a small volume. Thus, the biological sample may have a volume of between 2 μl and 500 μl, preferably between 3 μl and 400 μl, or between 3 μl and 100 μl, or else 5 μl and 50 μl. Such a volume of biological sample is in fact sufficient for the analysis on a routine instrument, but may be reduced on a remote biology device. This biological sample may be diluted, especially diluted in a buffer (such as Owren Koller buffer) or in plasma, before the addition of the solution of the substrate S and of the solution of the enzyme E.

In the examples presented at the end of this document, the biological sample has typically been diluted to represent 12.5% by volume of the final volume (i.e. 6.25 μl of sample in a final volume of 50 μl) before addition of the solution of the substrate S and of the solution of the enzyme E, which corresponds to approximately 1.8% by volume of the mixture to be tested having a total volume V of 350 μl, or else 50% by volume of the final volume (i.e. 25 μl of sample in a final volume of 50 μl), which corresponds to approximately 7.1% by volume of the mixture to be tested having a total volume V of 350 μl.

Preparation of the Sample to be Tested and Competing Reaction Conditions

The mixture to be tested is prepared by mixing the aliquot (diluted or undiluted) of the biological sample with a solution of the enzyme E and a solution of the substrate S to obtain a mixture of initial enzyme concentration [E]₀, or of initial activity A₀, and an initial substrate concentration [S]₀ in an order which is determined as a function of the nature of the inhibitor (see above). Generally speaking, the inhibition reaction and the enzymatic reaction are brought into competition under any suitable physicochemical experimental conditions (pH, temperature, ionic strength, etc.), identical to those used in the universal calibration method.

Step ii) (measuring the value of the detectable physical property of the label as a function of time and creating a curve having a rectilinear portion) and step iii) (calculating the gradient of the rectilinear portion of this curve in order to obtain the residual enzymatic activity in the stationary state) are carried out like steps a2) and a3) of the calibration method.

### Step 2) of the Assaying Method

Step 2) of the assaying method according to the invention, which consists in using the universal calibration curve obtained in step c) of the universal calibration method in order to determine the anti-enzyme activity of the biological sample from the residual enzymatic activity in the stationary state measured for the mixture to be tested, is carried out like step d2) of the method for creating the chart.

### Step 3) of the Assaying Method

In the cases wherein it is desired to obtain the assaying result in the form of an amount or of a concentration of inhibitor, the assaying method comprises the additional step 3), which consists in converting the anti-enzyme activity, expressed as a percentage and obtained in step 2), into the concentration of inhibitor using the chart (or standard curve) specific to the inhibitor, obtained in step d) of the universal calibration method.

This may be determined manually by plotting the anti-enzyme activity measured for the mixture to be tested on the graph and by determining the corresponding amount or concentration of inhibitor. Alternatively, the concentration of inhibitor may be calculated using the equation of the standard curve. For example, if the inhibitor is a reversible direct inhibitor, the concentration of inhibitor in the mixture to be tested may be calculated by equation 2 (or any other suitable equation) and, if the inhibitor is an irreversible indirect inhibitor, the concentration of inhibitor in the mixture to be tested may be calculated by equation 3 (or any other suitable equation).

At the end of step 3) of the assaying method of the invention, it is thus possible to determine the concentration of inhibitor present in the biological sample tested, for example the biological sample from a patient treated by the enzymatic inhibitor.

### Clinical Uses

In the field of direct oral anticoagulants (DOAs), a calibration/assaying method according to the invention may be used to assay a DOA in a biological sample from the patient treated by the DOA, with the aim of diagnosing an overdose, for example in the case of the patient having exceeded their dosage, in the case of drug interactions, in the case of changing treatment from an antivitamin K medicament (VKA) to a direct oral anticoagulant, or else before a surgical intervention or invasive procedure. Indeed, for example, if, after stopping administration of the DOA, the level of DOA is still high, there is a risk of hemorrhage for the patient during surgery and it is then necessary either to wait for the level of DOA to decrease, or to administer an anti-inhibitor compound to the patient in order to counteract the disadvantages of the inhibitor, using an antidote approach.

Thus, a subject of the present invention is the use of an assaying method according to the invention for estimating the hemorrhagic risk in a patient treated by DOA. In particular, a subject of the present invention is the use of an assaying method according to the invention for diagnosing an overdose in a patient treated by a DOA. Another subject of the invention is the use of an assaying method according to the invention for determining the hemorrhagic risk in a patient treated by DOA before a surgical intervention.

The present invention also relates to a method for estimating the hemorrhagic risk in a patient treated by a direct oral anticoagulant (for example a factor Xa inhibitor or a factor IIa inhibitor), the method comprising the following steps:
determining the amount or the concentration of direct oral anticoagulant in a biological sample from the patient, using an assaying method of the invention,
comparing this amount or concentration with a predetermined threshold, and
deeming there to be a hemorrhagic risk if the amount or concentration of direct oral anticoagulant is greater than the predetermined threshold, and
optionally determining the amount of anti-inhibitor compound to administer to the patient as a function of the amount or concentration of direct oral anticoagulant measured in the biological sample from the patient.

In certain embodiments, the patient treated by the DOA is suspected of having received an overdose of DOA. In other embodiments, the patient treated by the DOA has recently undergone a change in treatment from an antivitamin K medicament (VKA) to a direct oral anticoagulant. In yet other embodiments, the patient treated by the DOA is about to undergo a surgical intervention.

“Predetermined threshold” is intended here to mean an amount or concentration of inhibitor which is known in the art or which has been determined to correspond to an amount or concentration of inhibitor above which the inhibitor is in excess in the patient in a given situation (for example when there is an imminent surgical intervention). Such thresholds are fixed by the scientific and/or medical community for each DOA used clinically and are constantly being refined as more experience is gained in the clinical use of each DOA. Thus, for example, the decision threshold for allowing a surgical intervention in a patient taking a direct oral anticoagulant (DOA) treatment is a level less than or equal to 30 ng/ml (as a minimum for rivaroxaban, Pernod et al., Annales françaises d'anesthésie et de réanimation, 2013, 32(10): 691-700).

In a method according to the invention, the anti-inhibitor compounds which may be administered to patients on DOA with a hemorrhagic risk include hemostatic agents.

### B. Method for Screening Enzymatic Inhibitors

As indicated above, the calibration method according to the invention makes it possible to obtain results expressed as a percentage of anti-enzyme activity, a universal unit common to all the inhibitors of the calibrated enzyme, which makes it possible to compare the inhibitory efficiency of these inhibitors on the enzyme. The calibration method according to the invention may therefore be used in a screening method for identifying inhibitors of the enzyme.

Generally speaking, a screening method according to the invention comprises determining the anti-enzyme activity measured for a given concentration of a test compound and comparing this anti-enzyme activity with the anti-enzyme activity measured under the same conditions for the same concentration of a known inhibitor of the enzyme.

The present invention therefore also relates to a screening method for identifying an inhibitor of an enzyme, which comprises the following steps:
1) determining the residual enzymatic activity in the stationary state for a mixture of reaction medium M_{R}, of total volume V′″, and containing a test compound at an initial concentration [C], the enzyme E at an initial concentration [E]₀ or at an initial activity A₀, and a labeled substrate S specific to the enzyme at an initial concentration [S]₀, wherein:
   the substrate specific to the enzyme is labeled with a label having a detectable physical property,
   the enzyme is present in the mixture in excess relative to the test compound, and
   the residual enzymatic activity in the stationary state of the mixture is determined by the following steps:
   i) mixing the test compound with a solution of the enzyme E and a solution of the substrate S to obtain a mixture of initial enzyme concentration [E]₀, or of enzyme activity A₀, of initial substrate concentration [S]₀ and of concentration [C] of test compound,
   ii) measuring the value of the detectable physical property of the label and plotting, on a graph, the value of this physical property as a function of time in order to obtain a curve, the curve having a rectilinear portion corresponding to the stationary state, and
   iii) calculating the gradient of the rectilinear portion of the curve obtained in step ii),
   wherein the gradient obtained in step iii) is the residual enzymatic activity in the stationary state of the compound to be tested;
2) using the universal calibration curve obtained in step c) of the universal calibration method in order to determine the anti-enzyme activity of the test compound from the residual enzymatic activity in the stationary state measured for the mixture; and
3) comparing the anti-enzyme activity of the test compound determined in step 2) with the anti-enzyme activity determined under the same conditions for a standard inhibitor of the enzyme at a concentration [C], or comparing the anti-enzyme activity of the test compound determined in step 2) with a predetermined threshold, wherein the test compound is identified as an inhibitor of the enzyme if the anti-enzyme activity of the test compound is greater than the anti-enzyme activity of the standard inhibitor of the enzyme or if the anti-enzyme activity of the test compound is greater than the predetermined threshold.

### Step 1) of the Screening Method

Step 1) of the screening method according to the invention, which consists in determining the residual enzymatic activity in the stationary state for a mixture comprising the test compound, the same enzyme E and the labeled substrate S specific to the enzyme, is carried out similarly to step 1) of the assaying method.

The volume V′″ of the mixture containing the test compound may or may not be identical to the volume V of the mixtures used in steps a)-c) of the calibration method, and may or may not be identical to the volume V′ of the standardization mixtures used in step d) of the calibration method. Preferably, the volume V′″ is identical to the volume V and to the volume V′. The reaction medium M_{R} of the mixture containing the test compound is preferably identical or substantially identical to the reaction medium of the calibration samples and of the standardization samples.

Enzymes

As already indicated, the present invention may be applied to any enzyme (as defined above) for which it is beneficial to identify the inhibitors, for example with the aim of developing human or veterinary therapeutic inhibitors. Thus, in a calibration/screening method according to the invention, the enzyme is an identified therapeutic target. More specifically, in a calibration/screening method according to the invention, the enzyme may be any enzyme belonging to the class of the hydrolases, of the lyases or of the isomerases and identified as a therapeutic target.

Examples of lyases identified as therapeutic targets include, but are not limited to, DOPA decarboxylase (or aromatic L-amino acid decarboxylase, an enzyme, the known clinical inhibitors of which include carbidopa and benserazide, which are used in the context of Parkinson's disease, and methyldopa, which is used in the treatment of gestational hypertension); carbonic anhydrase (an enzyme present at the intracellular plasma membrane surface of red blood cells, and the clinical inhibitors of which are used as anti-glaucoma agents, diuretics, antiepileptics, and also in the treatment of gastroduodenal ulcers, neurological disorders or osteoporosis); histidine decarboxylase (the known clinical inhibitors of which, such as catechin and tritoqualine, are used as antihistamines); and ornithine decarboxylase (a known clinical inhibitor of which, eflornithine, is used in cancer treatment).

Examples of hydrolases identified as therapeutic targets include, but are not limited to, viral aspartyl proteases (or aspartic proteases) (the known clinical inhibitors of which include, but are not limited to, saquinavir and indinavir which are used in the treatment of HIV infection, and telaprevir and baceprevir which are used in the treatment of hepatitis C); serine proteases, such as human serine proteases which comprise pancreatic digestive enzymes such as trypsin, chymotrypsin and elastase (the inhibitors of which may be used in the treatment of serious diseases such as emphysema, cystic fibrosis or the development and progression of cancer), enzymes involved in coagulation such as thrombin, kallikrein, factor Xa (see above), and enzymes involved in fibrinolysis such as plasmin; metalloproteases which include, but are not limited to, human angiotensin-converting enzyme (see above), HMG-CoA reductase (the known clinical inhibitors of which include statins which are used as medicaments for lowering blood cholesterol levels), human enkephalinase (the inhibitors of which are used as analgesics, antidepressants, tranquilizers, and antidiarrheals); and other hydrolases, such as, for example, esterases, viral glycosidases, human glycosidases, lipases, phosphatases and phosphorylases.

Examples of isomerases identified as therapeutic targets include, but are not limited to, alanine isomerase (which is above all present in bacteria, which makes it a favorable target for the development of antibacterial agents).

Test Compounds

In a screening method according to the invention, any type of compound may be tested. Thus, a test compound may be a natural product or a synthetic product; it may be a single molecule or else a mixture or a complex of different molecules.

In certain embodiments, a test compound belongs to a chemical library (that is to say a library of molecules). Chemical libraries may contain several tens to several millions of chemical compounds. Chemical libraries of natural compounds in the form of bacterial or fungal extracts or in the form of plant extracts are available for example from Pan Laboratories (Bothell, Wash.) or MycoSearch (Durham, N.C.). Chemical libraries of synthetic compounds are also commercially available, for example from Comgenex (Princeton, N.J.), Brandon Associates (Merrimack, N.H.), Microsource (New Milford, Conn.), and Aldrich (Milwaukee, Wis.) or from large chemical companies such as Merck, Glaxo Welcome, Bristol-Myers Squibb, Novartis, Monsanto/Searle, and Pharmacia UpJohn.

The test compounds may belong to any class of molecules, such as proteins, peptides, peptidomimetics, peptoids, saccharides, steroids, etc. The test compounds may also be small molecules, or molecules of low molecular weight, generally of between approximately 50 and approximately 2500 dalton, such as, for example, between 500 and 700 dalton, and less than 350 dalton.

Operating Conditions

The operating conditions described for step a) of the universal calibration or for step 1) of the assaying method are applicable to step 1) of the screening method.

In certain procedures, a single compound is tested by an identification method of the present invention. In other procedures, several compounds are tested in parallel. In this case, the enzymatic reactions may be carried out on a multi-well plate, making it possible to conduct several tests simultaneously. Among the typical supports, there are microtitration plates and more particularly 12-, 24-, 48-, 96-, or 384- (or more) well plates, which are easy to handle.

A compound may be tested at a single concentration. Alternatively, a compound may be tested at several concentrations, for example concentrations within the range of concentrations wherein the known inhibitor of the enzyme, which is used as control, is active.

Identification of Compounds that Inhibit the Enzyme

In a screening method according to the invention, a test compound is identified as an inhibitor of the enzyme if the anti-enzyme activity of the test compound is greater than the anti-enzyme activity measured under the same conditions for a standard inhibitor of the enzyme, or if the anti-enzyme activity of the test compound is greater than the predetermined threshold.

“Standard inhibitor of the enzyme” denotes here a known inhibitor of the enzyme. Those skilled in the art know how to select the standard inhibitor to identify the test compounds liable to be of clinical interest. For example, the standard inhibitor may be an enzymatic inhibitor used clinically.

“Predetermined threshold” is intended here to mean an amount or concentration of inhibitor which, at the concentration tested, was identified as the value for anti-enzyme activity above which the test compound may be considered to be an inhibitor of the enzyme liable to be of clinical interest.

### Development of Therapeutic Inhibitors

The aim of a screening process according to the invention is to identify compounds that inhibit the enzyme and that are liable to be of clinical interest. A screening test according to the invention may be followed by other tests, for example by one or more screening tests according to the invention carried out in comparison with other known inhibitors of the enzyme and/or with other enzymes belonging to the same class. Alternatively or additionally, a screening test according to the invention may be followed by in vivo toxicity studies on cell models or on animal models. When a test compound has been identified as having an inhibitory activity on the enzyme, structure-activity relationship studies may be carried out with the aim of identifying new underlying inhibitor structures having improved properties compared to the test compound identified.

### III—Kits

The present invention also targets kits comprising equipment of use for carrying out a method according to the invention. In particular, the present invention relates to kits for assaying inhibitors of an enzyme and kits for screening compounds capable of inhibiting an enzyme. A kit is generally designed for a given enzyme. However, a kit may also be designed for more than one enzyme. For example, a kit may be designed for at least two enzymes involved in a shared biological mechanism, especially for at least two enzymes of blood coagulation. When the kit is intended to be used in an assaying method, it may be designed to be used in the case of a single inhibitor of the enzyme. Alternatively, the kit may be designed for assaying several different inhibitors of the enzyme (for example 2 different inhibitors or more than 2 different inhibitors). Moreover, when the kit is intended to be used in a method for assaying an inhibitor, the kit may be designed to enable a single assay. Alternatively, the kit may be designed to carry out a finite number of assays, for example 2 assays or 5 assays or 10 assays or 20 assays or 50 assays, etc.

Generally speaking, a kit according to the invention comprises the enzyme and the specific substrate. In certain embodiments, the specific substrate contained in the kit is labeled. In other embodiments, the specific substrate contained in the kit is not labeled but the kit comprises at least one reagent required for the labeling thereof. The enzyme and the substrate are provided in a suitable amount for carrying out the calibration and the envisaged number of assays.

When the kit is intended to be used in a method for assaying an inhibitor of the enzyme, the kit may comprise one or more samples for standardization of the inhibitor. When the kit is intended to be used for assaying different inhibitors of the enzyme, the kit may comprise one or more samples for standardization of each of the different inhibitors of the enzyme.

When the kit is intended to be used in a screening method for identifying inhibitors of the enzyme, the kit may comprise at least one standard inhibitor of the enzyme.

A kit according to the invention may also comprise reagents or solutions for carrying out a calibration/assaying method or a calibration/screening method according to the invention, for example reagents or solutions for diluting the biological sample, etc. In particular, a kit may comprise a buffer for diluting the biological samples, for example an OKB buffer. Protocols for using these reagents and/or solutions may also be included in the kit.

A kit according to the invention may also comprise quality controls.

The different components of the kit may be provided in solid form (for example in lyophilized form) or in liquid form. A kit may optionally comprise a container containing each of the reagents or solutions and/or containers for carrying out certain steps of a calibration/assaying method or calibration/screening method of the invention.

Generally speaking, a kit according to the invention also comprises instructions for carrying out a calibration/assaying method or calibration/screening method according to the invention.

A kit according to the invention may also comprise instructions in the form required by a government agency regulating the preparation, sale and use of biological products.

Unless they have been defined in another way, all the technical and scientific terms used here have the same meaning as that commonly understood by an ordinary specialist in the field to which this invention belongs. Likewise, all the publications, patent applications, all the patents and all other references mentioned here are incorporated by reference.

### Examples

The following examples and figures describe certain embodiments of the present invention. However, it will be appreciated that the examples are only presented by way of illustration and in no way limit the scope of the invention.

### FIGURE LEGENDS

FIG. 1: Universal calibrations. The graphs give the anti-Xa activity (%) as a function of the corresponding OD/min observed at 10 experimental points. (A) Broad-range methodology: the linear regression of these experimental points gives the equation of the straight line y=1.053−0.999 x with a coefficient of determination R²=0.994. (B) Narrow-range methodology: the linear regression of these experimental points gives the equation of the straight line y=1.034−1.081 x with a coefficient of determination R²=0.996.

FIG. 2: Conversion charts. These charts make it possible to convert the anti-Xa activity (%) measured into concentration expressed in ng/ml for each of the three direct oral anticoagulants (DOAs). (A) Broad-range methodology: non-linear regressions of equation 2 at several experimental points give, respectively, for rivaroxaban (●) i=0.73 and e=16.43, for apixaban (▴) i=1.35 and e=14.66 and for edoxaban (●) i=1.14 and e=15.66. (B) Narrow-range methodology: non-linear regressions of equation 2 at several experimental points give, respectively, for rivaroxaban (●) i=1.42 and e=15.36, for apixaban (▴) i=2.91 and e=13.23 and for edoxaban (●) i=1.98 and e=14.96.

FIG. 3: Rivaroxaban. (A) Broad-range methodology: the comparisons of the results of the assays of the levels of rivaroxaban measured on 20 overloads using the universal calibration principle according to the invention to the theoretical levels give the equation of the straight line y=6.91+0.98 x with a coefficient of determination R²=0.998. (B) Narrow-range methodology: the comparisons of the results of the assays of the levels of rivaroxaban measured on 8 overloads using the universal calibration principle according to the invention to the theoretical levels give the equation of the straight line y=1.63+0.96 x with a coefficient of determination R²=0.995.

FIG. 4: Apixaban. (A) Broad-range methodology: the comparisons of the results of the assays of the levels of apixaban measured on 24 overloads using the universal calibration principle according to the invention to the theoretical levels give the equation of the straight line y=10.41+0.97 x with a coefficient of determination R²=0.999. (B) Narrow-range methodology: the comparisons of the results of the assays of the levels of apixaban measured on 10 overloads using the universal calibration principle according to the invention to the theoretical levels give the equation of the straight line y=2.79+0.97 x with a coefficient of determination R²=0.999.

FIG. 5: Edoxaban. (A) Broad-range methodology: the comparisons of the results of the assays of the levels of edoxaban measured on 24 overloads using the universal calibration principle according to the invention to the theoretical levels give the equation of the straight line y=0.99 x+0.83 with a coefficient of determination R²=0.996. (B) Narrow-range methodology: the comparisons of the results of the assays of the levels of edoxaban measured on 10 overloads using the universal calibration principle according to the invention to the theoretical levels give the equation of the straight line y=1.00x−0.24 with a coefficient of determination R²=0.997.

FIG. 6: Improving the regression of the universal calibration. The graphs present the anti-Xa activity (%) as a function of the corresponding OD/min observed at 10 experimental points using narrow-range methodology. (A) Linear regression: the linear regression of these experimental points gives the straight line with the equation: y=1.034−1.081 x with a coefficient of determination R²=0.9963. (B) Regression with a second-order polynomial: the regression of these experimental points gives the polynomial of equation y=0.978−0.789 x−0.281 x² with a coefficient of determination R²=0.9998.

FIG. 7: Dilution in plasma—universal calibrations. The graphs present the anti-Xa activity (%) as a function of the corresponding OD/min observed at 10 experimental points using broad-range (●) and narrow-range (▴) methodologies. (A) Dilution in buffer: the linear regressions give, in broad-range methodology (●), the equation of the straight line y=1.053−0.999 x with a coefficient of determination R²=0.994 and, in narrow-range methodology (▴), the equation of the straight line y=1.034−1.081 x with a coefficient of determination R²=0.996. (B) Dilution in plasma: the linear regressions give, respectively, in broad-range methodology (●), the equation of the straight line y=1.022−1.173 x with a coefficient of determination R²=0.998 and, in narrow-range methodology (▴), the equation of the straight line y=1.020−1.198 x with a coefficient of determination R²=0.998.

FIG. 8: Dilution in plasma: rivaroxaban. (A) Broad-range methodology: the comparisons of the results of the assays of the levels of rivaroxaban measured on 20 overloads using the universal calibration principle according to the invention with dilution in plasma to the theoretical levels give the equation of the straight line y=1.03+0.96 x with a coefficient of determination R²=0.999. (B) Narrow-range methodology: the comparisons of the results of the assays of the levels of rivaroxaban measured on 8 overloads using the universal calibration principle according to the invention with dilution in plasma to the theoretical levels give the equation of the straight line y=1.15+0.95 x with a coefficient of determination R²=0.995.

FIG. 9: Universal calibration. The graph gives the anti-Xa activity (%) as a function of the corresponding OD/min observed at 10 experimental points. The linear regression of these experimental points gave the equation of the straight line y=0.094−0.518 x with a coefficient of determination R²=0.996.

FIG. 10: Conversion charts. The charts presented in this figure make it possible to convert the anti-Xa activity (%) measured into concentration in IU/ml for (A) unfractionated heparin: the linear regression of the experimental points gave the equation of the straight line y=2.354 x−0.02569 with a coefficient of determination R²=0.992, and (B) low-molecular-weight heparin: the linear regression of these experimental points gave the equation of the straight line y=1.621 x−0.02827 with a coefficient of determination R²=0.979.

FIG. 11: (A) Unfractionated Heparin. The comparison of the results of the assays of the levels of unfractionated heparin measured on 11 overloads using the universal calibration principle to the theoretical levels gave the equation of the straight line y=1.025 x−0.01107 with a coefficient of determination R²=0.979. (B) Low-Molecular-Weight Heparin. The comparison of the results of the assays of the levels of low-molecular-weight heparin measured on 7 overloads using the universal calibration principle to the theoretical levels gave the equation of the straight line y=0.9764 x+0.01489 with a coefficient of determination R²=0.985.

### EXAMPLE 1: REVERSIBLE DIRECT INHIBITORS

Vitamin K antagonists (VKA) are historically the first and only class of anticoagulants that are administered orally. The large amount of variability in the response of patients to this type of treatment, to which can be added numerous food and drug interactions, necessitate regular in vitro monitoring of the treatment, involving numerous blood samples being taken from the patient. This observation led the pharmaceutical industry to develop a new family of anticoagulants referred to as direct oral anticoagulants (DOAs) which, theoretically, do not require regular monitoring and only have relatively few food and drug interactions. This family of anticoagulants is split into two classes:
anti-Xas, direct inhibitors of factor Xa; and
anti-IIas, direct inhibitors of thrombin.

As indicated above, there are certain situations wherein assaying direct oral anticoagulants (DOAs) is useful, such as, for example, prior to an emergency surgical intervention, in suspected cases of overdose, or in the case of hemorrhage of unknown origin. The study presented here focuses on the family of the anti-Xas, the main molecules of which are:
rivaroxaban, sold by Bayer/Janssen Pharmaceutical under the name Xarelto®;
apixaban, sold by Bristol-Myers Squibb/Pfizer under the name Eliquis®; and
edoxaban, sold by Daiichi Sankyo under the name Savaysa® or Lixiana®.

### I. Experimental Protocols

### 1. Materials

The plasma samples (Etablissement Francais du Sang, La Plaine Saint-Denis, France) originated from healthy patients who were not following procoagulant or anticoagulant treatment. Different pools of plasma, dated January 2011, March 2012, April 2014, November 2014 and February 2015, were used for the experiments. Before producing the pools from plasma bags, tests were carried out in order to verify that the values of the prothrombin levels (PT), cephalin times with activator (CTA) and also the concentrations of coagulation factors were consistent. The plasma bags were subsequently thawed for 50 minutes at 37° C. and left at room temperature for 30 minutes to stabilize. A pool was produced and the plasma was agitated at 153 revolutions/min for 35 minutes before being divided up. The fractions were stored at approximately −70° C. Before use, they were thawed at 37° C. for 5 minutes.

The overloads of anticoagulant were produced from solutions of apixaban (Eliquis, Bristol-Myers Squibb, New York, United States) concentrated at 400 μg/ml, solutions of edoxaban tosylate (Lixiana, Daiichi-Sankyo, Tokyo, Japan) concentrated at 500 μg/ml and solutions of rivaroxaban (Xarelto, Bayer, Leverkusen, Germany) concentrated at 393 μg/ml.

The reagents from the commercial kit STA®—Liquid Anti-Xa (Diagnostica Stago, Asniéres sur Seine, France), F.Xa (factor Xa of bovine origin) and substrate (MAPA-Gly-Arg-pNA) were used. These commercial reagents and also the pool of normal human plasma Pool Norm (Diagnostica Stago, Asnieres sur Seine, France), the Owren Koller buffer (OKB) (pH 7.35, STA®—Owren-Koller, Diagnostica Stago, Asnieres sur Seine, France) and the desorption solution (DU) STA®—Desorb U (Diagnostica Stago, Asnieres sur Seine, France) were regenerated at room temperature for 30 minutes, as recommended by the manufacturer.

Dimethylsulfoxide (DMSO) (Carlo Erba, Val de Rueil, France) diluted to 5% in demineralized water was used for the experiments.

All the measurements were carried out on the STA® automated device (Diagnostica Stago, Asnieres sur Seine, France) AUT00460 (Hasting number). The software version for the automated device used in these experiments was version v3.04.05.

### 2. Methods

Anti-Xa Enzymatic Assays.

The kinetics were monitored by colorimetry at 405 nm, detecting the release of para-nitroanilide (pNA). The optical density (OD) was measured every two seconds. Two methodologies were optimized for assaying the three direct oral anti-Xa anticoagulants: the broad-range methodology and the narrow-range methodology. This is because the broad-range methodology makes it possible to assay the anticoagulants over the whole of the desired range; however, since the measurement of low concentrations of these anticoagulants requires great precision a narrow-range methodology was therefore developed.

In broad range, 6.25 μl of biological sample (plasma) were diluted with OKB in a final volume of 50 μl. The microcuvette was incubated for 240 seconds at 37° C. in the presence of 150 μl of substrate. The reaction was started by the addition of 150 μl of F.Xa, incubated beforehand at 37° C. In narrow range, 25 μl of sample were diluted with 25 μl of OKB. The experimental conditions for addition of substrate and enzyme are the same as for the broad-range methodology. The gradients were determined between 50 and 80 seconds.

Universal Calibration.

The calibration was carried out from a range of F.Xa composed of 10 points ranging from 10% to 100% F.Xa, corresponding to an anti-Xa activity ranging from 90% to 0%. The range was created from reagents from the kit STA® Liquid Anti-Xa. The F.Xa (2 ml, 1.8 ml, . . . , 0.2 ml) was diluted in OKB (0 ml, 0.2 ml . . . 1.8 ml) for a final volume of 2 ml. The pool of healthy plasmas dated 04/2014 was used for this experiment. 5% DMSO was diluted to 1/20th in the plasma. The results (measurements of the OD/min between 50 and 80 seconds) were determined in two replicates.

The gradients of the kinetics measured for the 10 calibration points make it possible to plot the graph of anti-Xa activity (%) as a function of the OD/min. The equation of the line obtained is used to convert the OD/min of the samples tested into anti-Xa activity as percentage. The equation of the line is given by a polynomial regression of first order or of second order.

Creation of the Conversion Charts.

The parameters of the chart, specific to each direct oral anticoagulant (DOA), were determined for both of the methodologies (broad-range methodology and narrow-range methodology) by a non-linear regression of equation 2 on a few experimental points. In broad range, the samples overloaded at 0, 100, 200, 300 and 400 ng/ml of DOA were used to create the chart associated with rivaroxaban. The samples overloaded at 0, 100, 200, 300, 400 and 500 ng/ml were used to create the charts associated with apixaban and with edoxaban. In narrow range, the samples overloaded at 0, 30, 65, and 100 ng/ml of DOA were used to create the chart associated with rivaroxaban. The samples overloaded at 0, 50, 100, and 150 ng/ml of DOA were used to create the charts associated with apixaban and with edoxaban. The OD/min was measured for each sample between 50 and 80 seconds.

Plasma Overloads.

The pool of plasma dated 04/2014 was used to produce the overloads. The stock solutions of direct oral anticoagulants (DOAs) were diluted in DMSO %. An intermediate solution at 10 μg/ml was used in order to produce the dilution ranges for the different overloads. The dilution of the DOAs in the plasma was to 1/20th. The concentrations of the overloads were 0, 10, 20, 30, 40, 50, 65, 75, 100, 125, 150, 175, 200, 225, 250, 275, 300, 325, 350, 375, 400 ng/ml for rivaroxaban and 0, 10, 20, 30, 40, 50, 75, 100, 125, 150, 175, 200, 225, 250, 275, 300, 325, 350, 375, 400, 425, 450, 475, 500 ng/ml for apixaban and edoxaban. The final volume of the overloads was 1 ml. The solutions containing edoxaban were stored in darkness. The results (measurements of the OD/min between 50 and 80 seconds) were determined for each overload in three replicates.

Configuration of the Tests for the Universal Calibration.

A test configuration was created by F.Xa dilution for the broad-range and narrow-range methodologies. The experimental conditions are the same as those described in the section “anti-Xa enzymatic assays”.

Configuration of the Tests for the Creation of the Charts.

As above, the different measurements were obtained according to the broad-range and narrow-range methodologies described in the section “anti-Xa enzymatic assays”.

Configuration of the Tests for the Plasma Overloads.

The measurements on the plasmas overloaded with DOAs were carried out according to the broad-range and narrow-range methodologies described in the section “anti-Xa enzymatic assays”.

Determining the Limit of Blank.

The limit of blank (LoB) was determined according to a standard procedure. The five different plasmas used were those dated January 2011, March 2012, April 2014, November 2014 and February 2015. Four replicates were carried out each day for three days, on the same automated device. The tests were carried out for the broad-range and narrow-range methodologies.

Determining the Limit of Detection.

The limit of detection (LoD) was determined according to a standard procedure. Low-analyte plasmas were prepared. The overloads were carried out to 1/20th. The plasma dated 04/2014 was used. When the regression of the universal calibration was a first-order polynomial, the concentrations of analytes in broad range were 10 ng/ml of rivaroxaban and of apixaban and 15 ng/ml for edoxaban. In narrow range, the concentrations of the overloads were 5 ng/ml for the three anticoagulants. Five overloads were produced for a final volume of 900 μl. When the regression of the universal calibration was a second-order polynomial, the concentrations of analytes in broad range and in narrow range were 2 ng/ml for the three direct oral anticoagulants (DOAs). The final volume of the overloads was 1000 μl. Each overload was separated into three tubes. The samples were frozen at −70° C. until their use. The analyses were carried out over three days on the same automated device with four analytical replicates per day and per overload.

Anti-Xa Enzymatic Assays with Dilution in Plasma.

The experimental conditions are identical to those described in the preceding sections, except for the fact that the dilution was not carried out in buffer but in plasma. The diluent used was the Pool Norm. The universal calibration was nonetheless obtained by diluting F.Xa in OKB under the same conditions as those described in the section “Universal calibrations”.

### II. Results

### 1. Universal Calibrations

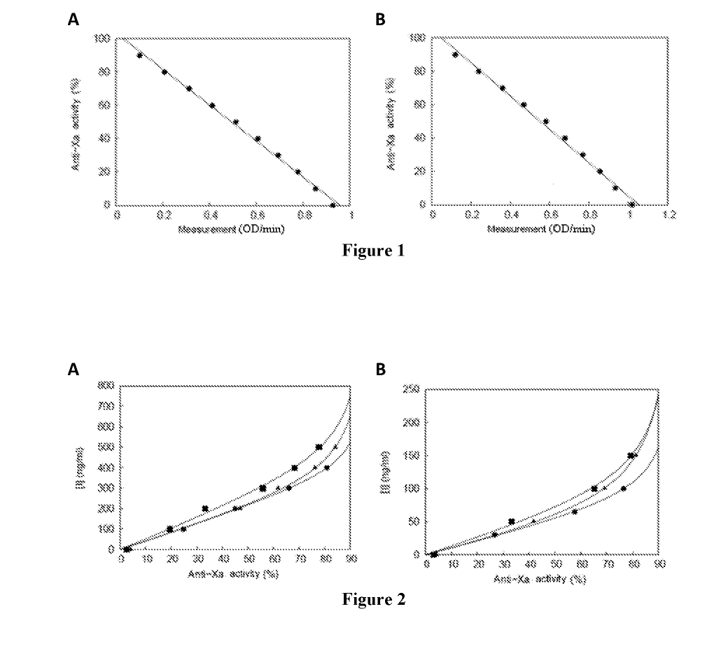

FIG. 1 presents examples of universal calibrations according to the invention, obtained respectively in broad-range and narrow-range methodologies, which express the results in anti-Xa activity (%) as a function of the OD/min measured. The linear regressions of the different experimental points give the equations:

y=1.053−0.999x(R²=0.994), and y=1.034−1.081x(R=0.996).

The theoretical equation of these universal calibrations is given by the straight line of equation 1 as described above in the description; the results obtained here are in perfect agreement with this expression, that is to say that a linear regression is obtained having a coefficient of determination very close to 1 and an intercept point close to 1. It is interesting to note that the equations of these two straight lines are different. This is explained by the fact that the dilution of the sample is different between the two methodologies (1/8 for the broad-range methodology and 1/2 for the narrow-range methodology); a matrix effect is observed.

### 2. Conversion Charts

FIG. 2 illustrates the charts which make it possible to convert the anti-Xa activity (%) measured into concentration expressed in ng/ml for each of the three direct oral anticoagulants (DOAs). These charts were created by carrying out a non-linear regression of equation 2 on samples for which the pairs (anti-Xa activity, concentration of anticoagulant) were known. In broad-range methodology, these non-linear regressions have, respectively, coefficients of determination R² of 0.997 for rivaroxaban, 0.995 for apixaban and 0.993 for edoxaban. In narrow-range methodology, these non-linear regressions have, respectively, coefficients of determination R² of 0.993 for rivaroxaban, 0.997 for apixaban and 0.992 for edoxaban. All these coefficient of determination values highlight the relevance of the mathematical model developed. In addition, at equal concentrations, rivaroxaban has the highest in vitro anti-Xa activity whereas edoxaban has the lowest anti-Xa activity: this confirms that the concentration expressed in ng/ml is not a universal unit for assaying anti-Xa DOAs.

3. Assays of Plasmas Overloaded with Direct Oral Anticoagulants (DOAs)

In this section, the results of the assays of the levels of plasmas overloaded with DOAs (rivaroxaban, apixaban and edoxaban) were obtained using the universal calibration principle according to the invention and compared to the theoretical levels. The results are deemed to be satisfactory when the gradient of the linear regression is between 0.9 and 1.1 and when the coefficient of determination R² is greater than or equal to 0.95.

Rivaroxaban.

FIG. 3 presents the results of the comparisons of the assays of the rivaroxaban levels measured using the universal calibration principle in broad-range methodology (20 overloads) and in narrow-range methodology (8 overloads) and compared to the theoretical levels. These comparisons gave, respectively, a gradient of 0.98 and 0.96 and a coefficient of determination of 0.998 and 0.995, thereby validating the results.

Apixaban.

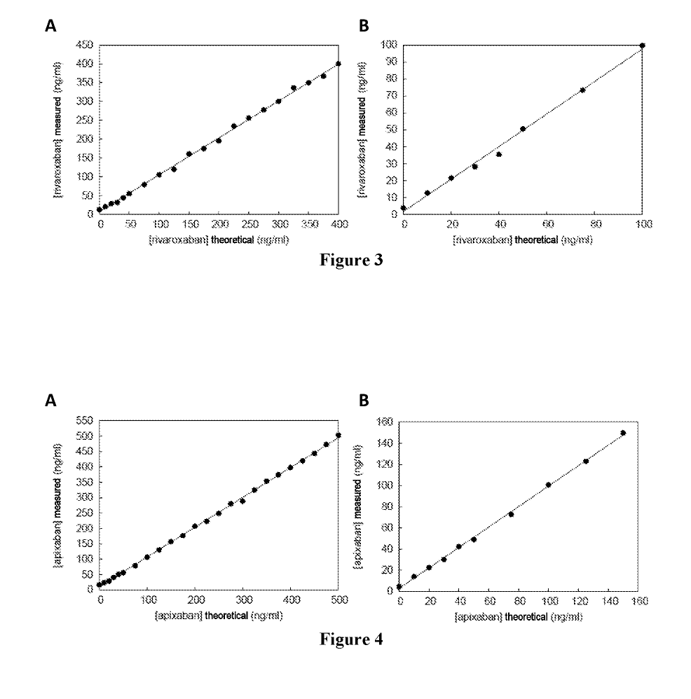

FIG. 4 presents the results of the comparisons of the assays of the apixaban levels measured using the universal calibration principle in broad-range methodology (24 overloads) and in narrow-range methodology (10 overloads) and compared to the theoretical levels. These comparisons gave, respectively, a gradient of 0.97 and 0.97 and a coefficient of determination of 0.999 and 0.999, thereby validating the results.

Edoxaban.

FIG. 5 presents the results of the comparisons of the assays of the edoxaban levels measured using the universal calibration principle in broad-range methodology (24 overloads) and in narrow-range methodology (10 overloads) and compared to the theoretical levels. These comparisons give, respectively, a gradient of 0.99 and 1.00 and a coefficient of determination of 0.996 and 0.997, thereby validating the results.

### 4. Limits of Blank and Limits of Detection

In this section, the limits of blank (LoB) and the limits of detection (LoD) measured were obtained using the universal calibration principle in broad-range and narrow-range methodology. Moreover, a method for improving these values in narrow-range methodology is proposed. As a reminder, the decision threshold for allowing a surgical intervention in a patient taking a direct oral anticoagulant (DOA) treatment is a level less than or equal to 30 ng/ml (as a minimum for rivaroxaban, Pernod et al., Annales françaises d'anesthésie et de reanimation, 2013, 32(10): 691-700).

Broad-Range Methodology.

Table 1 below lists the limits of blank and the limits of detection obtained in broad-range methodology using the universal calibration principle according to the invention. The LoD values observed were respectively 21.5 ng/ml for rivaroxaban, 21.8 ng/ml for apixaban and 28.8 ng/ml for edoxaban. These results indicate that the broad-range methodology does not make it possible to measure low levels of anti-Xa DOAs with the precision required in the context of an emergency surgical intervention.

TABLE 1
Broad-range methodology - limits of blank and limits of detection.

| | LoB (ng/ml) | LoD (ng/ml) |
|---|---|---|
| rivaroxaban | 16.897 | 21.496 |
| apixaban | 16.613 | 21.723 |
| edoxaban | 20.802 | 28.734 |

Narrow-Range Methodology.

Table 2 below lists the limits of blank and the limits of detection obtained in narrow-range methodology using the universal calibration principle according to the invention. The LoD values observed were respectively 6.9 ng/ml for rivaroxaban, 6.9 ng/ml for apixaban and 8.7 ng/ml for edoxaban. These results demonstrate that the narrow-range methodology has sufficiently good performance to precisely measure very low levels of anti-Xa DOAs.

TABLE 2
Narrow-range methodology - limits
of blank and limits of detection.

| | LoB (ng/ml) | LoD (ng/ml) |
|---|---|---|
| rivaroxaban | 5.352 | 6.849 |
| apixaban | 5.450 | 6.882 |
| edoxaban | 6.802 | 8.657 |

Optimization in Narrow-Range Methodology.

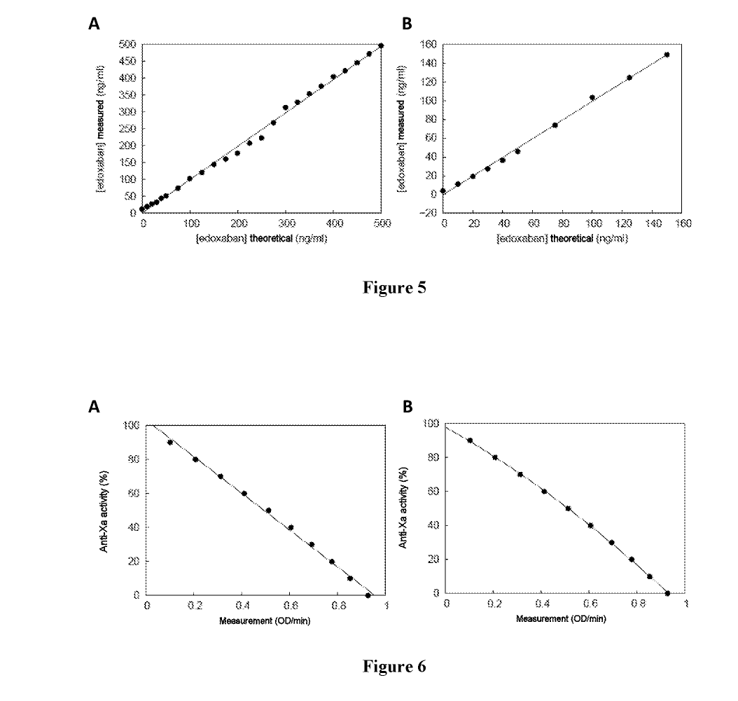

It has been demonstrated and validated experimentally that the universal calibration follows a straight line given by equation 1. However, empirically, a regression of the universal calibration by a second-order polynomial should express the experimental points better than a linear regression. This is confirmed experimentally—see FIG. 6. The regression of the universal calibration by a second-order polynomial makes it possible to improve the LoB and LoD in narrow-range methodology (cf. table 3 below) but does not impact the quality of the comparisons of the levels measured to the theoretical levels (results not shown).

TABLE 3
Optimization in narrow-range methodology -
limits of blank and limits of detection.

| | LoB (ng/ml) | LoD (ng/ml) |
|---|---|---|
| rivaroxaban | 2.854 | 4.202 |
| apixaban | 2.759 | 4.001 |
| edoxaban | 3.574 | 4.955 |

### 5. Dilution in Plasma

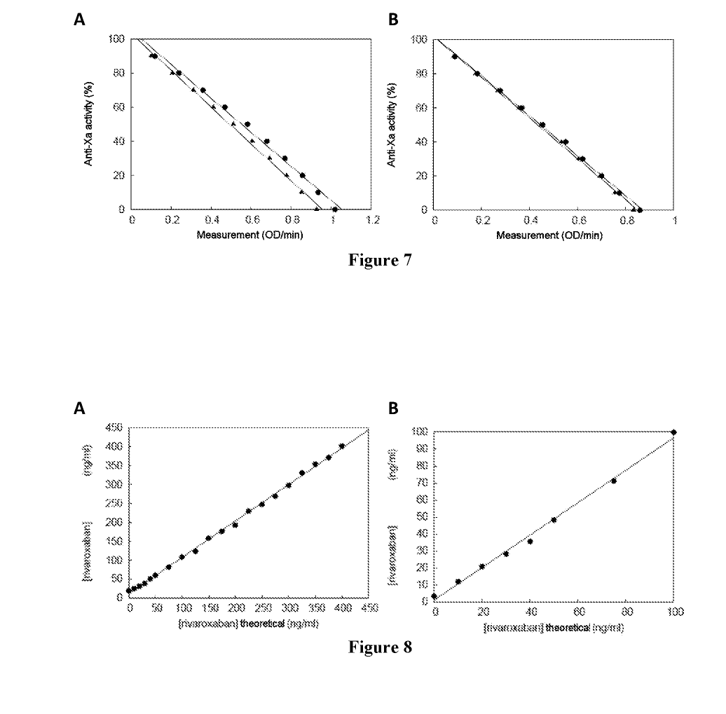

The difference in the sample dilution value between the broad-range methodology (dilution=1/8) and narrow-range methodology (dilution=1/2) induces a matrix effect. A method is proposed here in order to overcome this. For this purpose, in the universal calibration, the sample is no longer diluted in buffer (OKB) but it is diluted in plasma (Pool Norm) with the factors of dilution remaining unchanged. FIG. 7 compares the appearance of the universal calibrations when the sample is diluted in buffer to the appearance of the universal calibrations when the sample is diluted in plasma. The linear regressions of the experimental points in broad-range methodology and in narrow-range methodology give, respectively, the equations of straight lines:

y=1.053−0.999x and

y=1.034−1.081x+

when the sample is diluted in buffer, and the equations of straight lines:

y=1.022−1.173x and

y=1.020−1.198x

when the sample is diluted in plasma.

These results indicate that, when the sample is diluted in plasma, the equations of the two straight lines of the universal calibrations are superimposed on one another. In this configuration, it is no longer necessary to carry out two calibrations but simply a single one that is common to the two methodologies (broad-range methodology and narrow-range methodology). It should also be highlighted that the comparison of the theoretical levels of rivaroxaban to the levels measured in overloaded plasmas using the universal calibration principle with dilution of the sample in plasma gives a gradient of 0.96 and a coefficient of determination of 0.999 in broad-range methodology and a gradient of 0.95 and a coefficient of determination of 0.995 in narrow-range methodology, see FIG. 8: these results demonstrate that it is possible to measure in vitro the levels of direct oral anticoagulants in samples diluted in plasma.

### EXAMPLE 2: IRREVERSIBLE INDIRECT INHIBITORS

### I. Experimental Protocols

### 1. Materials

The plasma samples (Etablissement Francais du Sang, La Plaine Saint-Denis, France) and the reagents in example 2 are the same as those used in example 1.

The overloads of heparins were produced from solutions of heparin calcium (Cari-Parine®, Sanofi-Aventis, Paris, France), concentrated at 5000 IU/0.2 ml and of tinzaparin sodium (Innohep®, Leo Pharma, Ballerup, Denmark) at 10 000 IU/0.5 ml.

### 2. Methods

Anti-Xa Enzymatic Assays.

The kinetics were monitored as in example 1. The methodology was optimized for the assay of unfractionated heparins and low-molecular-weight heparins. Two (2) μl of plasma were diluted in OKB in a final volume of 100 ml. The microcuvette was incubated for 690 seconds at 37° C. in the presence of 100 ml of F.Xa. The measurement phase was triggered by the addition of 100 ml of substrate, incubated beforehand at 37° C. The gradients were determined between 20 and 50 seconds.

Universal Calibration.

The calibration was carried out as in example 1, except for the fact that the measurements were carried out between 20 and 50 seconds.

Creation of the Conversion Charts.

The parameters of the chart, specific to each heparin, were determined by a linear regression of equation 3 on a few experimental points. The samples overloaded at 0, 0.3, 0.6, and 1.0 IU/ml were used to create the chart associated with heparin calcium. The samples overloaded at 0, 0.2, 0.4, and 0.6 IU/ml were used to create the chart associated with tinzaparin sodium. The OD/min is measured for each sample between 20 and 50 seconds.

Plasma Overloads.

A pool of plasma was used to produce the overloads. The stock solutions of heparins were diluted in physiological serum. The concentrations of the overloads were 0, 0.1, 0.2, 0.3, 0.4, 0.5, 0.6, 0.7, 0.8, 0.9, and 1.0 IU/ml for heparin calcium and 0, 0.1, 0.2, 0.3, 0.4, 0.5, and 0.6 IU/ml for tinzaparin sodium. The final volume of the overloads was 0.5 ml. The results (measurements of the OD/min between 20 and 50 seconds) were determined for each overload in three replicates.

Configuration of the Tests for the Universal Calibration, Configuration of the Tests for Creating the Charts, and Configuration of the Tests for the Plasma Overloads.

The experimental conditions are the same as those described in the section “anti-Xa enzymatic assays”.

### II. Results

### 1. Universal Calibration

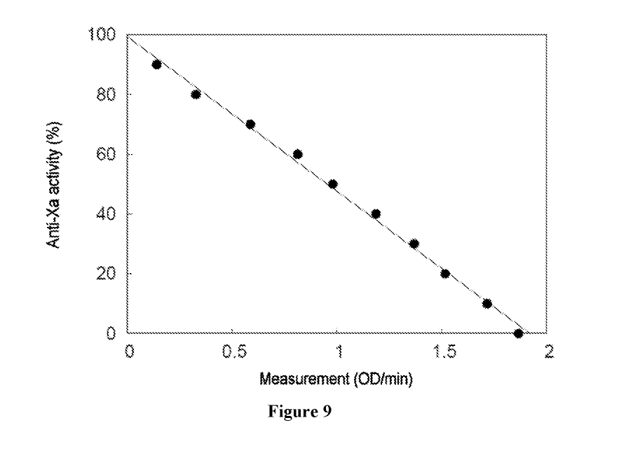

FIG. 9 illustrates the universal calibration which expresses the results in anti-Xa activity (%) as a function of the OD/min measured. The linear regression of the different experimental points gives the equation y=0.994−0.518 x=(R²=0.996). The theoretical equation of this calibration is given by the straight line of equation 1. The results obtained are in perfect agreement with this expression, that is to say a linear regression with a coefficient of determination very close to 1 and an intercept point close to 1.

### 2. Conversion Charts

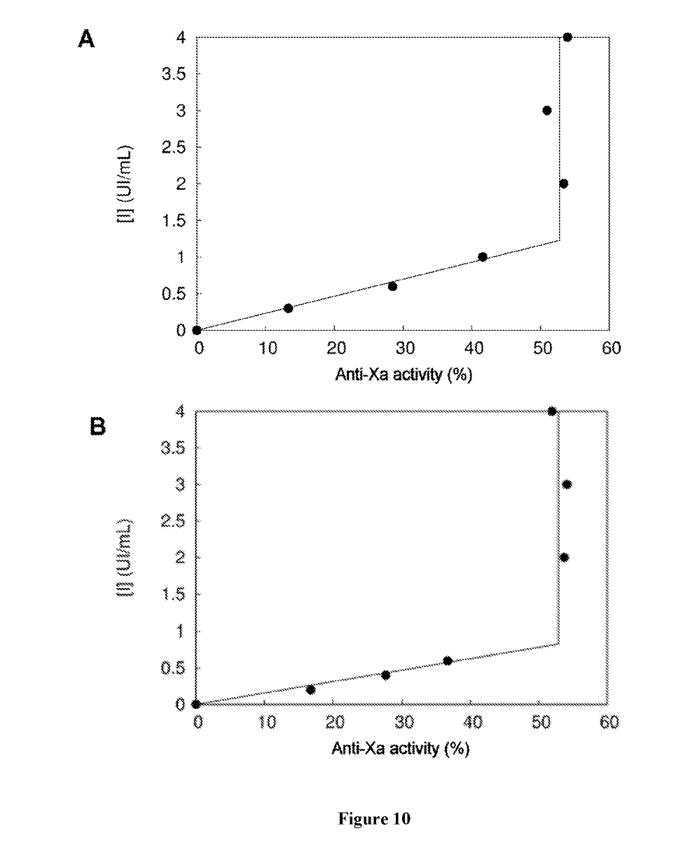

FIG. 10 presents the charts created for converting the anti-Xa activity (%) measured into concentration in IU§ /ml for each of the two families of heparins. These charts were created by carrying out a linear regression on samples for which the pairs (anti-Xa activity, concentration of anticoagulant) were known. The linear regressions of the experimental points gave the equation of the straight line y=2.354 x−0.01569 for unfractionated heparin and the equation of the straight line y=1.621 x−0.028247 for low-molecular-weight heparin. These equations are in perfect harmony (straight line with an intercept close to zero) with equation 2. Moreover, equation 3 is theoretically valid when the heparin concentration is less than or equal to the antithrombin concentration. In the opposite case, the heparin is no longer measurable, given that it completely saturates the plasma antithrombin. This behavior can be seen in the charts of FIG. 10. Indeed, the linear regression only applies to anti-Xa activities less than or equal to 52.75% for unfractionated heparin and 52.85% for low-molecular-weight heparin. These values are extremely consistent, although they were determined independently. Thus, all the results obtained confirm the relevance of the mathematical model developed here.

3. Assays of Plasmas Overloaded with Heparins

The results of the assays of the levels of plasmas overloaded with unfractionated heparin and with low-molecular-weight heparin obtained using the universal calibration principle according to the present invention are presented here by comparison to the theoretical levels. The results were deemed to be satisfactory when the gradient of the linear regression is between 0.9 and 1.1 and when the coefficient of determination R² is greater than or equal to 0.95.

Unfractionated Heparin.

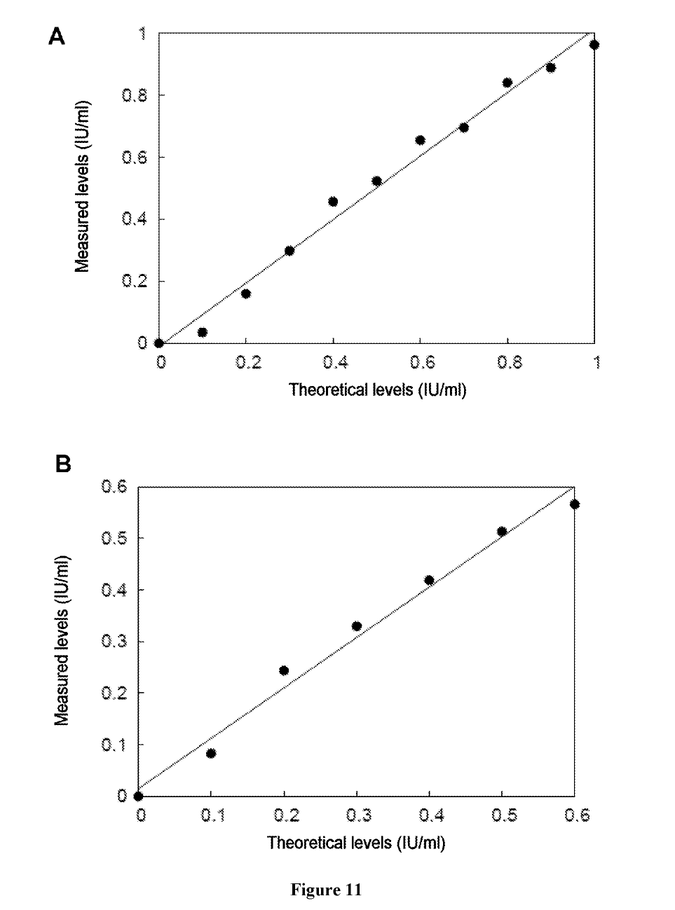

FIG. 11(A) presents the results obtained using 11 unfractionated heparin overloads. The comparison of the levels measured using the universal calibration principle to the theoretical levels gave a gradient of 1.025 and a coefficient of determination of 0.987, which validates the results.

Low-Molecular-Weight Heparin.

FIG. 11(B) presents the results obtained using 7 low-molecular-weight heparin overloads. The comparison of the levels measured using the universal calibration principle to the theoretical levels gave a gradient of 0.9764 and a coefficient of determination of 0.985, which validates the results.

1. A method for obtaining a universal calibration for assaying an inhibitor of an enzyme, the method comprising the following steps:
a) determining the residual enzymatic activity in the stationary state for each of a plurality of mixtures containing the enzyme E and a substrate S that is labeled and specific to the enzyme, wherein:
the substrate specific to the enzyme is labeled with a label having a detectable physical property,
in each of the mixtures, the substrate is present in excess relative to the enzyme,
the mixtures contain the same initial substrate concentration, [S]₀,
the mixtures have the same total volume V and the same reaction medium M_{R},
the mixtures have known and decreasing initial enzyme concentrations, the highest initial enzyme concentration being [E]₀, or have known and decreasing initial enzyme activities, the highest initial enzyme activity being A₀, and
the residual enzymatic activity in the stationary state of a mixture is determined by the following steps:
a1) mixing a solution of the enzyme E and a solution of the substrate S to obtain a mixture of known initial enzyme concentration, or of known initial enzyme activity, and of initial substrate concentration [S]₀,
a2) measuring the value of the detectable physical property of the label and plotting, on a graph, the value of this physical property as a function of time in order to obtain a curve, the curve having a rectilinear portion corresponding to the stationary state, and
a3) calculating the gradient of the rectilinear portion of the curve obtained in a2),
wherein the gradient obtained in a3) is the residual enzymatic activity in the stationary state of the mixture;
b) for each of the mixtures of step a), converting the initial enzyme concentration of the mixture into anti-enzyme activity expressed as a percentage by standardizing said initial enzyme concentration of the mixture relative to the highest initial enzyme concentration [E]₀, or converting the initial enzyme activity of the mixture into anti-enzyme activity expressed as a percentage by standardizing said initial enzyme activity of the mixture relative to the highest initial enzyme activity A₀,
c) creating a universal calibration curve by plotting, on a graph, for each mixture, the anti-enzyme activity determined in step b) as a function of the residual enzymatic activity in the stationary state obtained in step a).

2. The method according to claim 1, wherein the enzyme belongs to the class of hydrolases, to the class of lyases, or to the class of isomerases.

3. The method according to claim 1, wherein the inhibitor is a reversible direct inhibitor, a reversible indirect inhibitor, an irreversible direct inhibitor or an irreversible indirect inhibitor.

4. The method for obtaining a universal calibration according to claim 1, wherein in step b), for a mixture with an initial enzyme concentration [E], the anti-enzyme activity expressed as a percentage is calculated by the equation:
AntiEnzyme_{%}=1−([E]/[E]₀),
and, for a mixture with an initial enzyme activity A, the anti-enzyme activity expressed as a percentage is calculated by the equation:
AntiEnzyme_{%}=1−(A/A₀).

5. The method for obtaining a universal calibration as according to claim 1, wherein, in step c), the calibration curve is a straight line with the equation:
$${AntiEnzyme}_{( \% )} = 1 - ( \frac{1}{{v}_{0}} \times v )$$
wherein:
AntiEnzyme_{(%)} is the anti-enzyme activity expressed as a percentage,
v is the residual enzymatic activity in the stationary state,
1/v₀ is the gradient of the calibration curve, and v₀ is the residual enzymatic activity in the stationary state observed in the absence of an inhibitor.

6. The method for obtaining a universal calibration according to claim 1, further comprising a step d) consisting in creating a conversion chart specific to an inhibitor of the enzyme E and which makes it possible to convert the anti-enzyme activity, determined for a sample to be tested, into the amount or concentration of inhibitor.

7. The method for obtaining a universal calibration according to claim 6, wherein step d) comprises the following sub-steps:
d1) determining the residual enzymatic activity in the stationary state for at least two standardization mixtures each containing the inhibitor at a known initial concentration, the enzyme E at the initial concentration [E]₀ or at the initial activity A₀, and the labeled substrate S specific to the enzyme at the concentration [S]₀, wherein:
in each of the standardization mixtures, the enzyme is present in excess relative to the inhibitor,
the standardization mixtures have the same volume V′ and the same reaction medium M_{R}, and
the residual enzymatic activity in the stationary state of a mixture is determined by the following steps:
d1′) mixing the inhibitor with a solution of the enzyme E and a solution of the substrate S in order to obtain a standardization mixture with a known initial concentration of inhibitor,
d1″) measuring the value of the detectable physical property of the label and plotting, on a graph, the value of this physical property as a function of time in order to obtain a curve, the curve having a rectilinear portion corresponding to the stationary state, and
d1′″) calculating the gradient of the rectilinear portion of the curve obtained in d1″),
wherein the gradient obtained in step d1′″) is the residual enzymatic activity in the stationary state of the standardization mixture;
d2) for each of the standardization mixtures, using the universal calibration curve obtained in step c) of the universal calibration method in order to determine the anti-enzyme activity of the mixture from the residual enzymatic activity in the stationary state measured in step d1′″) for the standardization mixture; and
d3) creating a standard curve or chart, by plotting on a graph, for each standardization mixture, the initial concentration of inhibitor of the standardization mixture as a function of the anti-enzyme activity determined in step d2).

8. The method for obtaining a universal calibration according to claim 7, wherein the volume V′ is identical to the volume V.

9. The method for obtaining a universal calibration according to claim 7, wherein step d) also comprises the sub-step d4) consisting in determining the equation of the standard curve by a regression of the pairs (anti-enzyme activity, concentration of inhibitor) plotted on the graph in step d3).

10. The method for obtaining a universal calibration according to claim 7, wherein
if the inhibitor is a reversible direct inhibitor, the equation of the standard curve is the following equation:
$${[ I ]}_{0} = i \times \frac{{AntiEnzyme}_{( \% )}}{1 - {AntiEnzyme}_{( \% )}} + e \times {AntiEnzyme}_{( \% )}$$
wherein:
AntiEnzyme_{(%)} is the anti-enzyme activity,
[I]₀ is the concentration of inhibitor present in the sample to be tested, and
i and e are two fixed constants specific to the inhibitor; and
if the inhibitor is an irreversible indirect inhibitor, the equation of the standard curve is the following equation:
[I]₀=e×AntiEnzyme_{(%)}
wherein:
AntiEnzyme_{(%)} is the anti-enzyme activity in the sample to be tested,
[I]₀ is the concentration of inhibitor present in the sample, and
e is a fixed constant specific to the inhibitor.

11. A method for assaying an inhibitor of an enzyme in a biological sample, comprising the following steps:
1) determining the residual enzymatic activity in the stationary state for a mixture to be tested of reaction medium M_{R}, of volume V″, and containing an aliquot of the biological sample containing the inhibitor, the enzyme E at an initial concentration [E]₀ or at an initial activity A₀, and a labeled substrate S specific to the enzyme at an initial concentration [S]₀, wherein:
the substrate specific to the enzyme is labeled with a label having a detectable physical property, and
the residual enzymatic activity in the stationary state of the mixture to be tested is determined by the following steps:
i) mixing the aliquot of the biological sample with a solution of the enzyme E and a solution of the substrate S to obtain a mixture of initial enzyme concentration, or [E]₀, or of initial enzyme activity A₀, and an initial substrate concentration [S]₀,
ii) measuring the value of the detectable physical property of the label and plotting, on a graph, the value of this physical property as a function of time in order to obtain a curve, the curve having a rectilinear portion corresponding to the stationary state, and
iii) calculating the gradient of the rectilinear portion of the curve obtained in step ii),
wherein the gradient obtained in step iii) is the residual enzymatic activity in the stationary state of the mixture to be tested; and
2) using the universal calibration curve obtained in step c) of the calibration method according to claim 1 in order to determine the anti-enzyme activity of the biological sample from the residual enzymatic activity in the stationary state measured for the mixture to be tested.

12. The assaying method according to claim 11, wherein the biological sample is a sample of blood, of plasma, of platelet-rich plasma, of platelet-poor plasma, or of plasma containing platelet or erythrocyte microparticles or any other cell.

13. The assaying method according to claim 12, wherein the biological sample is a platelet-poor plasma sample.

14. The method according to claim 11, wherein the enzyme is an enzyme of blood coagulation selected from coagulation factors, kallikrein and plasmin, preferably selected from factor IIa, factor Xa and plasmin.

15. The method according to claim 14, wherein the inhibitor is selected from antithrombin, heparin cofactor II, alpha-2-macroglobulin, hirudin, lepirudin, desirudin, rivaroxaban, apixaban, edoxaban, betrixaban, dabigatran, bivalirudin, argatroban, unfractionated heparins, low-molecular-weight heparins, pentasaccharides and danaparoid sodium.

16. The assaying method according to claim 11, wherein the volume V″ is identical to the volume V.

17. The assaying method according to claim 11, further comprising the following step:
3) converting the anti-enzyme activity expressed as a percentage and obtained in step 2) into a concentration of inhibitor using the chart specific to the inhibitor which was obtained in step d) of the calibration method according to claim 6.

18. The assaying method according to claim 17, wherein the volume V″ is identical to the volume V′.

19. (canceled)

20. A method for estimating the hemorrhagic risk in a patient treated with a direct oral anticoagulant, the method comprising the following steps:
determining the amount or the concentration of direct oral anticoagulant in a biological sample from the patient, using an assaying method according to claim 11;
comparing this amount or concentration with a predetermined threshold; and
concluding that there is a hemorrhagic risk if the amount or concentration of direct oral anticoagulant is greater than the predetermined threshold.

21. The method for estimating the hemorrhagic risk in a patient treated with a direct oral anticoagulant according to claim 20, further comprising the following step:
determining the amount of anti-inhibitor compound to administer to the patient as a function of the amount or concentration of direct oral anticoagulant measured in the biological sample from the patient.

22. The method according to claim 20, wherein the patient treated with the direct oral anticoagulant is a patient suspected of having received an overdose of direct oral anticoagulant or is a patient recently having undergone a change in treatment from an antivitamin K medicament to the direct oral anticoagulant, or is a patient about to undergo a surgical intervention.

23. A screening method for identifying an inhibitor of an enzyme, comprising the following steps:
1) determining the residual enzymatic activity in the stationary state for a mixture of reaction medium M_{R}, of volume V′″, and containing a test compound at an initial concentration [C], the enzyme E at an initial concentration [E]₀ or at an initial activity A₀, and a labeled substrate S specific to the enzyme at an initial concentration [S]₀, wherein:
the substrate specific to the enzyme is labeled with a label having a detectable physical property,
the enzyme is present in the mixture in excess relative to the test compound, and
the residual enzymatic activity in the stationary state of the mixture is determined by the following steps:
i) mixing the test compound with a solution of the enzyme E and a solution of the substrate S to obtain a mixture of initial enzyme concentration [E]₀, or of enzyme activity A₀, an initial substrate concentration [S]₀ and a concentration [C] of test compound,
ii) measuring the value of the detectable physical property of the label and plotting, on a graph, the value of this physical property as a function of time in order to obtain a curve, the curve having a rectilinear portion corresponding to the stationary state, and
iii) calculating the gradient of the rectilinear portion of the curve obtained in step ii),
wherein the gradient obtained in step iii) is the residual enzymatic activity in the stationary state of the compound to be tested;
2) using the universal calibration curve obtained in step c) of the universal calibration method in order to determine the anti-enzyme activity of the test compound from the residual enzymatic activity in the stationary state measured for the mixture; and
3) comparing the anti-enzyme activity of the test compound determined in step 2) with the anti-enzyme activity determined under the same conditions for a standard inhibitor of the enzyme at a concentration [C], or comparing the anti-enzyme activity of the test compound determined in step 2) with a predetermined threshold, wherein the test compound is identified as an inhibitor of the enzyme if the anti-enzyme activity of the test compound is greater than the anti-enzyme activity of the standard inhibitor of the enzyme or if the anti-enzyme activity of the test compound is greater than the predetermined threshold.

24. The screening method according to claim 23, wherein the volume V′″ is identical to the volume V.

25. The screening method according to claim 23, wherein the enzyme belongs to the class of the hydrolases or to the class of the lyases.

26. A kit for identifying an inhibitor of an enzyme E, comprising
the enzyme E,
a labeled substrate S specific to the enzyme, and
instructions for carrying out a screening method according to claim 23.

27. A kit for assaying at least one of the inhibitors of an enzyme E in a biological sample, the kit comprising
the enzyme E,
a labeled substrate S specific to the enzyme,
at least one inhibitor of the enzyme, and
instructions for carrying out an assaying method according to claim 11.

28. The kit according to claim 27, further comprising at least one other inhibitor of the enzyme.
